SEQUENCE LISTING (36 sequences)

SEQ ID NO: 1 - DNA, 20 bp, synthetic construct
  source 1..20: mol_type = other DNA
ctggacctgttccgactgta

SEQ ID NO: 2 - DNA, 19 bp, synthetic construct
  source 1..19: mol_type = other DNA
caacgttcaggttggttgc

SEQ ID NO: 3 - DNA, 20 bp, synthetic construct
  source 1..20: mol_type = other DNA
cctcccagtcttgagtcagc

SEQ ID NO: 4 - DNA, 20 bp, synthetic construct
  source 1..20: mol_type = other DNA
cataggcgttctcagggtgt

SEQ ID NO: 5 - DNA, 20 bp, synthetic construct
  source 1..20: mol_type = other DNA
atcctgcaccaccaactgct

SEQ ID NO: 6 - DNA, 20 bp, synthetic construct
  source 1..20: mol_type = other DNA
gggccatccacagtcttctg

SEQ ID NO: 7 - DNA, 23 bp, synthetic construct
  source 1..23: mol_type = other DNA
tgatggattctaatcttccaagg

SEQ ID NO: 8 - DNA, 19 bp, synthetic construct
  source 1..19: mol_type = other DNA
gtgagccagctcagcaaac

SEQ ID NO: 9 - DNA, 20 bp, synthetic construct
  source 1..20: mol_type = other DNA
ctgctctcgaagctccaact

SEQ ID NO: 10 - DNA, 20 bp, synthetic construct
  source 1..20: mol_type = other DNA
tttccacagcgtttgaagaa

SEQ ID NO: 11 - DNA, 20 bp, synthetic construct
  source 1..20: mol_type = other DNA
acctgacctccgacacaaag

SEQ ID NO: 12 - DNA, 20 bp, synthetic construct
  source 1..20: mol_type = other DNA
tggcttctgttgcctcttct

SEQ ID NO: 13 - DNA, 20 bp, synthetic construct
  source 1..20: mol_type = other DNA
atctgtgtgcgctgtactgg

SEQ ID NO: 14 - DNA, 20 bp, synthetic construct
  source 1..20: mol_type = other DNA
gcgatgcaatttcctcattt

SEQ ID NO: 15 - DNA, 18 bp, synthetic construct
  source 1..18: mol_type = other DNA
ctgctgctcctgctgtcc

SEQ ID NO: 16 - DNA, 18 bp, synthetic construct
  source 1..18: mol_type = other DNA
cagcgctgttcgtcacat

SEQ ID NO: 17 - DNA, 20 bp, synthetic construct
  source 1..20: mol_type = other DNA
gcaaatgcaaacaatgcaaa

SEQ ID NO: 18 - DNA, 20 bp, synthetic construct
  source 1..20: mol_type = other DNA
cacttgtcggaagcctcttt

SEQ ID NO: 19 - DNA, 20 bp, synthetic construct
  source 1..20: mol_type = other DNA
gaagcaatgtcccagggtta

SEQ ID NO: 20 - DNA, 20 bp, synthetic construct
  source 1..20: mol_type = other DNA
ctcctcggtgagtcttctgg

SEQ ID NO: 21 - DNA, 20 bp, synthetic construct
  source 1..20: mol_type = other DNA
agcagattggcctcagtgtt

SEQ ID NO: 22 - DNA, 20 bp, synthetic construct
  source 1..20: mol_type = other DNA
tccatcactaggggttcctg

SEQ ID NO: 23 - DNA, 19 bp, synthetic construct
  source 1..19: mol_type = other DNA
gttgacatccgtaaagacc

SEQ ID NO: 24 - DNA, 18 bp, synthetic construct
  source 1..18: mol_type = other DNA
tggaaggtggacagtgag

SEQ ID NO: 25 - DNA, 20 bp, synthetic construct
  source 1..20: mol_type = other DNA
caggaggcagaagcagctat

SEQ ID NO: 26 - DNA, 21 bp, synthetic construct
  source 1..21: mol_type = other DNA
tggcttagaagacccttggtt

SEQ ID NO: 27 - DNA, 20 bp, synthetic construct
  source 1..20: mol_type = other DNA
tatatcatggccgacaagca

SEQ ID NO: 28 - DNA, 20 bp, synthetic construct
  source 1..20: mol_type = other DNA
actgggtgctcaggtagtgg

SEQ ID NO: 29 - DNA, 20 bp, synthetic construct
  source 1..20: mol_type = other DNA
gccttcaccccagagaaagg

SEQ ID NO: 30 - DNA, 21 bp, synthetic construct
  source 1..21: mol_type = other DNA
ccggtttggatttacatgttg

SEQ ID NO: 31 - DNA, 18 bp, synthetic construct
  source 1..18: mol_type = other DNA
gacaagagaaccatcgag

SEQ ID NO: 32 - DNA, 17 bp, synthetic construct
  source 1..17: mol_type = other DNA
ctgaaactcaccgacac

SEQ ID NO: 33 - DNA, 21 bp, synthetic construct
  source 1..21: mol_type = other DNA
gtggagtctactggtgtcttc

SEQ ID NO: 34 - DNA, 21 bp, synthetic construct
  source 1..21: mol_type = other DNA
gtgcaggaggcattgcttaca

SEQ ID NO: 35 - DNA, 19 bp, synthetic construct
  source 1..19: mol_type = other DNA
cccactgtcattgatgagg

SEQ ID NO: 36 - DNA, 24 bp, synthetic construct
  source 1..24: mol_type = other DNA
cgaaccctatctaaaacaaggtca

(19) United States
(12) Patent Application Publication
(10) Pub. No.: US 2025/0170273 A1
(43) Pub. Date: May 29, 2025

(54) THERAPEUTIC FACTORS FOR THE TREATMENT OF POLYQ DISEASES

(71) Applicants: UNIVERSITA' DEGLI STUDI DI PADOVA (95%), Padova (IT); UNIVERSITÄT WIEN (5%), Wien (AT)

(72) Inventors: Graziano Martello, Padova (IT); Giorgia Maria Ferlazzo, Verona (IT); Anna Maria Gambetta, Padova (IT); Martin Leeb, Vienna (AT)

(73) Assignees: UNIVERSITA' DEGLI STUDI DI PADOVA (95%), Padova (IT); UNIVERSITÄT WIEN (5%), Wien (AT)

(21) Appl. No.: 18/837,199

(22) PCT Filed: Feb. 9, 2023
(86) PCT No.: PCT/IB2023/051156

§ 371 Date: Aug. 9, 2024

(30) Foreign Application Priority Data
Feb. 11, 2022 (IT) 102022000002498

(51) Int. Cl. A61K 48/00; A61K 38/17; A61K 38/44; A61P 25/28

(52) U.S. Cl. CPC A61K 48/005; A61K 38/1709; A61K 38/177; A61K 38/44; A61P 25/28; C12N 2750/14122; C12N 2750/14143; C12N 2750/14145

(57) ABSTRACT

The present invention relates to therapeutic proteins (therapeutic proteins) for use in the treatment of polyQ diseases by therapy, mRNAs, gene therapy vectors or viral particles coding for said therapeutic proteins and composition comprising them.

The present invention relates to therapeutic proteins (therapeutic proteins) for use in the treatment of polyQ diseases by therapy, mRNAs, gene therapy vectors or viral particles coding for said therapeutic proteins and composition comprising them.

### STATE OF THE ART

The polyglutamine (polyQ) diseases are a group of neurodegenerative disorders caused by expanded cytosine-adenine-guanine (CAG) repeats encoding a long polyQ tract in the respective proteins. To date, at least nine polyQ disorders have been described: six spinocerebellar ataxias (SCA) types 1, 2, 6, 7, 17; Machado-Joseph disease (MJD/SCA3); Huntington's disease (HD); dentatorubral pallidoluysian atrophy (DRPLA); and spinal and bulbar muscular atrophy, X-linked 1 (SMAX1/SBMA).

Currently, there is neither a cure nor prevention for these diseases, and only symptomatic treatments for polyQ diseases exist. Long-term pharmacological treatment is so far disappointing, probably due to unwanted complications and decreasing drug efficacy.

Among polyQ diseases, Huntington's disease (HD) is the most widespread monogenic neurodegenerative disorder among the Caucasian population (prevalence of ˜7-11 individuals out of 100,000 people). This is due to its autosomal dominant inheritance, given that a single copy of the mutated HTT gene is sufficient to confer pathological phenotypes, both in patients and in experimental models.

The disease is caused by an abnormal expansion (>36) of a CAG triplet in the Huntingtin (HTT) gene, resulting in the formation of a mutant HTT protein, containing a polyQ repeat. The wild-type HTT protein includes from 9 to 35 Q residues at the NH2 terminus and has been implicated in the formation of the neural tube, in the resistance to apoptotic stimuli and in the transcriptional control of BDNF and related genes. Indeed, the polyglutamine-encoding CAG trinucleotide repeats expansion confers a toxic gain-of-function activity to mutant HTT, leading to abnormal accumulation of aggregation-prone proteins, increased sensitivity to glutamate toxicity, mitochondrial damage and misregulation of the transcriptional program. However, it is still hard to know which processes are early causative events and which are consequences. Moreover, HTT protein is ubiquitously expressed, yet, HD is characterized by cell-population specific damages, loss of efferent medium spiny neurons in the striatum of the basal ganglia and massive degeneration of cortical structures.

Despite all the acquired knowledge on HD pathogenesis, no effective therapeutic intervention is available yet. Even though ameliorating the cellular processes impaired in HD gave promising results in animal models, all clinical trials to date have not demonstrated efficacy. For this reason, there is a need in the art to find different therapeutic strategies for the treatment of polyQ diseases.

### SUMMARY OF THE INVENTION

The Authors of the present invention, focused on the identification of factors, i.e. therapeutic proteins able to reduce toxicity of polyQ mutated proteins causing polyQ diseases, i.e. counteracting cell death, oxidative stress and/or transcriptional alterations caused by said mutant polyQ expressed proteins.

The authors of the present invention have identified at least 9 genes that, when expressed in vitro and/or in vivo (in animal models) through suitable gene therapy means, such as a suitable viral vector (e.g. an AAV vector) or in the form of mRNA, strongly ameliorate the detrimental effects of the mutant HTT gene, thereby providing a novel therapeutic strategy against polyQ diseases. Said genes code for polypeptide molecules, i.e. proteins, herein defined as “therapeutic proteins” that, once expressed in a cell or in an animal expressing one or more polyQ mutated proteins causing a polyQ disease, share the functional feature of reducing the cellular toxicity caused by said polyQ mutated protein/s, thereby reducing the pathological effects of a mutated protein causing a polyQ disease (cfr. Table 1).

The invention relates to one or more therapeutic protein capable of reducing the toxicity of mutated proteins causing polyQ diseases, or nucleotide sequences coding for said therapeutic protein/s, for use in the treatment of said diseases, preferably by gene therapy (herein intended as gene delivery of said therapeutic protein/s).

Therefore, the invention relates to one or more therapeutic protein capable of reducing the toxicity of mutated protein expressed by one or more of ATN1, HTT, AR, ATXN1, ATXN2, ATXN3, CACNA1A, ATXN7, PPP2R2B, TBP genes, wherein said protein comprise a pathological number of polyQ residues (as reported in table 1) for use in the treatment of polyQ diseases, e.g. by gene therapy.

According to the invention polyQ diseases are, in conformity with the state of the art, DRPLA (Dentatorubropallidoluysian atrophy), HD (Huntington's disease), SBMA (Spinal and bulbar muscular atrophy), SCA1 (Spinocerebellar ataxia Type 1), SCA2 (Spinocerebellar ataxia Type 2), SCA3 (Spinocerebellar ataxia Type 3 or Machado-Joseph disease), SCA6 (Spinocerebellar ataxia Type 6), SCA7 (Spinocerebellar ataxia Type 7), SCA12 (Spinocerebellar ataxia Type 12), SCA17 (Spinocerebellar ataxia Type 17).

According to the invention, gene therapy is a therapeutic strategy comprising the delivery of a nucleic acid encoding a factor capable of treating a disease by arresting or modifying the progression of said disease.

Objects of the invention are
One or more therapeutic protein capable of reducing the toxicity of mutated proteins causing polyQ diseases, or nucleotide sequences coding for said therapeutic protein/s, for use in the treatment of said diseases, preferably by gene therapy;
A delivery system comprising a nucleotide sequence coding for the therapeutic proteins, isoforms or homologs as herein defined or claimed;
An expression vector or delivery system or a vector suitable for gene therapy, or an adeno-associated viral vector AAV or a Lentiviral vector, or a nanoparticle, or an LNPcomprising comprising one or more cDNA or RNA sequences coding for a therapeutic protein capable of reducing the toxicity of mutated proteins causing polyQ diseases;
A cDNA or mRNA molecule (free or complexed with one or more suitable carrier molecules), coding for a therapeutic protein capable of reducing the toxicity of mutated proteins causing polyQ diseases;
Said delivery system, or expression vector or cDNA molecule or mRNA molecule (free or complexed with one or more suitable carried molecules) for use in the treatment of a polyQ disease;

A method of treatment of a polyQ disease comprising administering to a subject in need thereof therapeutically effective amounts of:
a delivery system, an expression vector comprising a cDNA or RNA sequence coding for a therapeutic protein capable of reducing the toxicity of mutated proteins causing polyQ diseases as defined in any embodiment in the description or in the claims or of
a cDNA or mRNA molecule (free or in a suitable delivery system) coding for a therapeutic protein capable of reducing the toxicity of mutated proteins causing polyQ diseases as defined in any embodiment in the description or in the claims or of
a therapeutic protein capable of reducing the toxicity of mutated proteins causing polyQ diseases as defined in any embodiment of the description and in the claims.

In all the objects of the invention: 35
said mutated protein is expressed by one or more of ATN1, HTT, AR, ATXN1, ATXN2, ATXN3, CACNA1A, ATXN7, PPP2R2B, TBP genes, and comprises a pathological number of polyQ residues (as reported in table 1);
said one or more therapeutic protein, isoform or homolog thereof is capable of reducing the toxicity of mutated protein expressed by one or more of ATN1, HTT, AR, ATXN1, ATXN2, ATXN3, CACNA1A, ATXN7, PPP2R2B, TBP genes comprising a pathological number of polyQ residues (as reported in table 1);
said one or more therapeutic protein is selected from metal regulatory transcription factor 1 (gene: MTF1-mtf1), lysine-specific demethylase 5B (gene: KDM5B-Kdm5b), lysine-specific demethylase 2B (gene: KDM2B-Kdm2b), F-box only protein 34 (gene: FBX034-Fbx034), essential MCU regulator, mitochondrial (gene: SMDT1-Smdt1), Ephrin type-A receptor 4 (gene: EPHA4-Ephha4), Transducin-like enhancer protein 4 (gene: TLE4-Tle4), Arfaptin-1 (gene: ARFIP1-Arfip1), and AT-rich interactive domain-containing protein 5B (gene: ARID5B-Arid5B);
said isoforms or homologs retain the capability of the therapeutic protein of which they are isoforms or homologs as defined herein, of reducing the toxicity of the mutated protein causing a given polyQ disease.

### Glossary

Gene therapy according to the present description has the meaning commonly recognized in the art, it therefore refers to a therapy through transfer of genetic material (e.g replacing a mutated gene with a healthy copy, or inactivating a mutated gene functioning improperly, or introducing a new gene, such as a gene coding for a therapeutic protein according to the invention, into the body). in the subject in need of a treatment, i.e. the therapeutic delivery of nucleic acid into a patient's cells as a drug to treat disease. Gene therapy according to the art and to the present invention can be achieved by transferring the genetic material of interest in the subject in need of treatment using a mRNA molecule or a non-viral or a viral method. Viral expression vectors commonly used for human gene therapy include retroviruses, adenoviruses, lentiviruses, herpes simplex virus, vaccinia virus, and adeno-associated virus. Viral vector genomes are either incorporated in the host's genome or stay as episomes.

Adeno-associated viral (AAV) vectors according to the present description has the meaning commonly recognized in the art. AAV belongs to the parvoviruses. It is a single-stranded, non-enveloped DNA virus of 4.7 kb size, which causes a latent infection of human cells. Parvoviruses represent an alternative to malignancy-related retroviruses. Many naturally occurring AAV serotypes and variants have been isolated from various animal species including mammals, birds, and reptiles. Among them, the common AAV serotypes include AAV1, 2, 3, 4, 5, 6, 7, 8, 9, 10, 11, 12 and 13. Besides these serotypes, there are a number of AAV variants and mutants that have been used for AAV vector-mediated gene delivery, including AAV-DJ, AAV-LK03, AAV-PHP.B, AAV-PHP.eB, AAV-Retro, AAV2.7m8, among others.

Typical AAV vectors comprise two ITR (inverted terminal repeat) regions, and within said ITRs at least the following elements: a promoter, a gene of interest and a terminator/polyadenylation signal.

Optimal serotype for brain gene therapy according to the present description refers to the AAV serotype which is best known in the art as to be more effective and suitable for brain gene therapy, i.e. to serotypes displaying strong neural tropisms. According to the present description, hence, the optimal serotype will be the serotype displaying the strongest tropism and infection efficiency depending on the target brain cell for therapy.

Viral particle according to the present description indicates a viral capsid containing a nucleotide construct leading, upon introduction of a suitable cell, to the expression of the molecule of interest, the viral particle according to the invention is also called in the art “gene transfer vector”, and comprises a viral capsid containing a genetic construct that will be transferred into the infected cell.

A mRNA molecule according to the present description is a mRNA molecule suitable for therapy, i.e. a molecule comprising 3′ and 5′ UTR elements flanking the coding sequence, a 5′ Cap and a poly A tail.

According to the present description the term Polyglutamine disease, or PolyQ disease, is a genetic disorder caused by trinucleotide repeat expansion, a kind of mutation in which repeats of three nucleotides (trinucleotide repeats) increase in copy numbers until they cross a threshold above which they become unstable. PolyQ disorders are caused by a CAG repeat expansion in protein-coding portions of specific genes, in all cases, the expanded CAG repeats are translated into an uninterrupted sequence of glutamine residues, forming a polyQ tract, and the accumulation of polyQ proteins damages key cellular function.

Known PolyQ diseases in the present description are depicted in Table 1 and include DRPLA (Dentatorubropallidoluysian atrophy), HD (Huntington's disease), SBMA (Spinal and bulbar muscular atrophy), SCA1 (Spinocerebellar ataxia Type 1), SCA2 (Spinocerebellar ataxia Type 2), SCA3 (Spinocerebellar ataxia Type 3 or Machado-Joseph disease), SCA6 (Spinocerebellar ataxia Type 6), SCA7 (Spinocerebellar ataxia Type 7), SCA12 (Spinocerebellar ataxia Type 12), SCA17 (Spinocerebellar ataxia Type 17).

PolyQ mutated protein according to the present description refers to a mutated protein comprising an anomalous number of Q residues (increased) due to an expansion of the triplet coding for said amino acid in the gene coding for said protein with respect to the wild type protein, wherein the mutated protein is pathogenic and causes a polyQ disease. The additional Q residues in polyQ mutated proteins are a cluster of additional Q residues resulting in a strand of Q residues with a relevant increase in their number with respect to the wild type protein. The average number of additional Q residues in polyQ mutated proteins is known in the art, an example is reported in Table 1.

PolyQ mutated proteins according to the present description are listed in Table 1.

In the present description the expression “capable of reducing the toxicity” referred to the therapeutic protein or isoforms or homologs thereof, means that the expression of the therapeutic protein object of the invention in a model animal of a polyQ disease or in model cells of polyQ diseases (i.e. model animals or cells expressing the polyQ mutant protein causing a polyQ disease) reduces the death of cells in said models compared to the observed cell death of said models when the therapeutic protein object of the invention is not expressed and/or reduces oxidative stress in said models compared to the observed oxidative stress of said models when the therapeutic protein object of the invention is not expressed and/or reduces transcriptional alterations in said models compared to the observed transcriptional alterations of said models when the therapeutic protein object of the invention is not expressed.

According to the present description a therapeutic protein capable of reducing the toxicity of the mutated protein causing a given polyQ disease is a protein exerting a therapeutic effect (i.e. a therapeutic protein) following administration to a subject in need thereof or expression in said subject through gene therapy.

According to the present description, the expression, “capable of reducing the toxicity of mutated proteins causing polyQ disease” means that said therapeutic protein or expression of said nucleotide sequence, is “capable of reducing the toxicity of said mutated protein causing said polyQ disease upon introduction into a mammalian cell expressing a mutated protein causing a polyQ disease or upon administration to a patient affected by a polyQ disease”.

According to the present description the expression “toxicity of polyQ mutated proteins” comprises cell death, oxidative stress and transcriptional alterations caused by said mutant polyQ expressed proteins.

A pathogenic mutated protein causing a polyQ disease is a protein comprising an increased number of Q residues in the form of a polyQ sequence as indicated in Table 1.

In any part of the description and of the claims, the expression “for use” in a treatment encompasses also the use of one or more therapeutic proteins (including isoforms or homologs), constructs, vectors, mRNA as defined in the description or in the claims for the preparation of a medicament for said treatment, wherein said one or more therapeutic proteins (including isoforms or homologs), constructs, vectors, mRNAs are formulated with one or more suitable excipients and/or carriers into a medicament that can be administered for the treatment of polyQ diseases as illustrated in the present description.

### DETAILED DESCRIPTION OF THE FIGURES

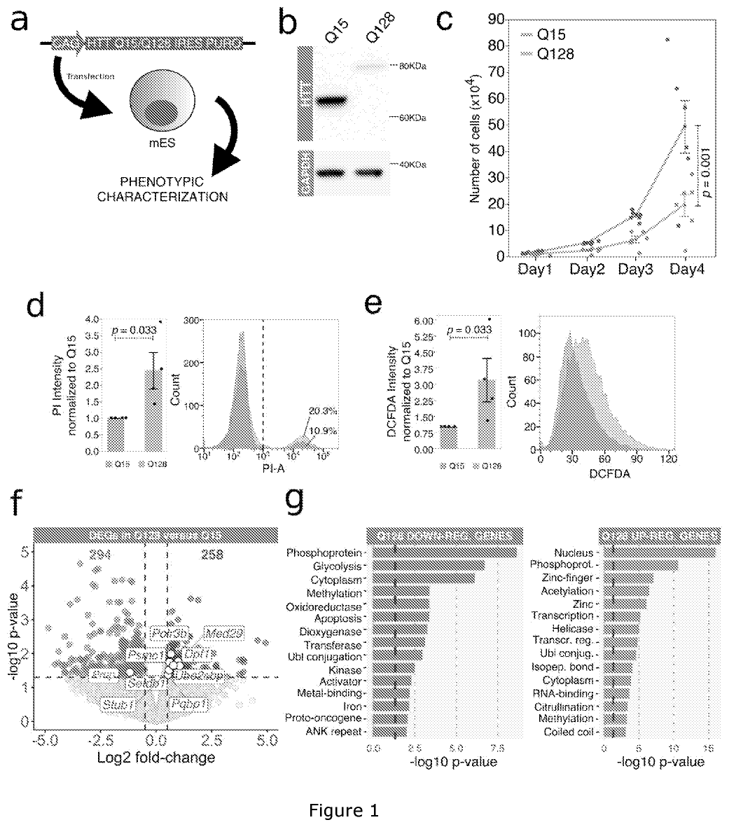

FIG. 1—Establishment and characterization of mutant HTT-expressing ES cells. a, Experimental strategy for generation and characterization of wild-type ES cells (Rex1GFP-d2) expressing N-terminal fragment of either mutant (128 CAG repeats) or wild-type (15 CAG repeats) HTT by DNA transfection and puromycin selection, named Q15 and Q128 cells, respectively. b, Western Blot of HTT confirmed the correct production of a 80 kDa and a 65 kDa form of HTT protein in Q128 and Q15 cells respectively. Q128 HTT protein expression resulted lower compared to Q15 HTT. GAPDH was used as loading control. c, Proliferation assay of Q128 (orange) and Q15 (blue) ES cells showed pronounced impairment in cell proliferation due to mutant HTT expression. Bars indicate the mean±SEM of 6 independent experiments, shown as dots. p-values were calculated with Two-way Repeated Measure ANOVA.

d, Measurement of cell death by Propidium Iodide uptake and Flow Cytometry. Q128 cells (orange) display higher cell death, compared to Q15 (blue) cells. The fraction of PI-positive cells (dashed line) was calculated for each sample and fold-changes were calculated relative to the Q15 samples. Bars indicate the mean±SEM of 4 independent experiments shown as dots. Representative flow cytometry plots are shown on the right. p-values were calculated with One-tailed one sample Mann-Whitney U test. e, Measurement of 2′,7′-dichlorofluorescin diacetate (DCFDA) median fluorescence as an evaluation of Reactive Oxygen Species production in Q15 (blue) versus Q128 (orange) cells. Bars indicate the mean±SEM of 4 independent experiments shown as dots. Fold-changes were calculated relative to the Q15 samples. Representative flow cytometry profiles of ROS detection are represented in the right panel. p-values were calculated with One-tailed one sample Mann-Whitney U test. f, Transcriptome analysis of Q128 and Q15 cells. DOWN-regulated (Log 2 fold-change <0.5 and p-value <0.05) and UP-regulated (Log 2 fold-change >0.5 and p-value <0.05) genes are indicated in blue and red, respectively. Known genes previously associated with Huntington's disease were highlighted. Thresholds used are indicated by dashed lines. p-values were calculated with Wald Test with no adjustment. g, Gene list enrichment analysis of DOWN-regulated genes (left panel, blue bars) and UP-regulated genes (right panel, red bars) revealed a statistically significant enrichment (p-value=0.05, shown as a black dashed line) for genes involved in metabolism (phosphorylation, glycolysis), proteasome (apoptosis, ubiquitin conjugation) and transcription in general. p-values were calculated by Fisher Exact test using DAVID database.

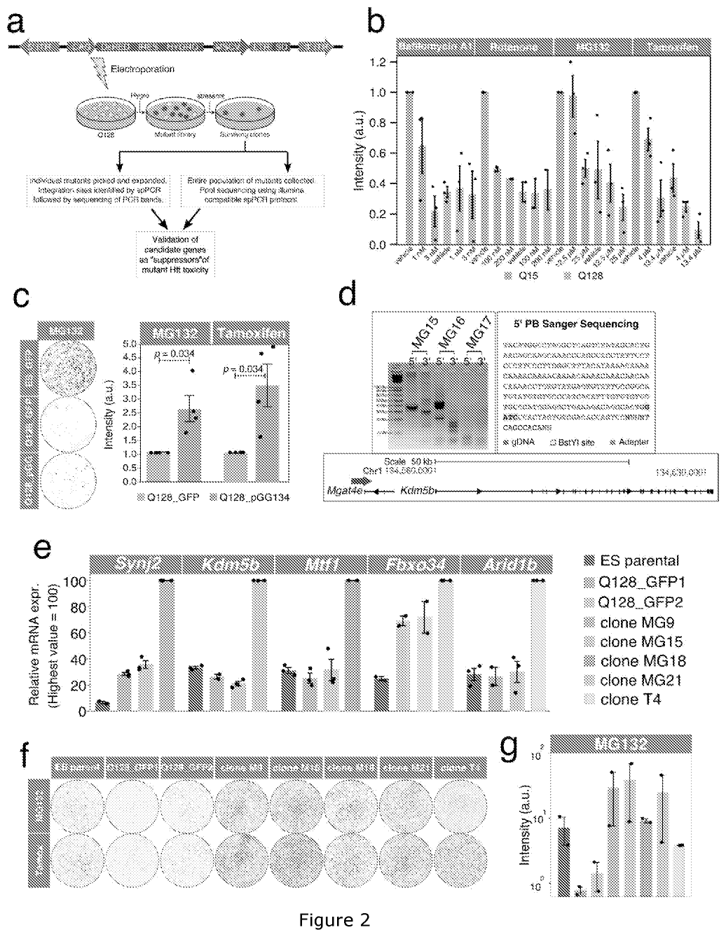

FIG. 2—A gain-of-function screen for suppressors of mutant HTT toxicity. a, Top panel: diagram of the piggyBac vector pGG134 containing MSCV 5′LTR followed by a splice donor site from exon 1 of mouse Foxf2 gene, resulting in the activation of genes flanking the site of integration. ITRs allow random integration at TTAA sites and the DsRed-IRES-Hygromycin cassette was used to identify cells with stable vector integration. Bottom panel: a schematic diagram of the screening strategy used to identify new proteins involved in HTT-dependent toxicity.

Electroporation of pGG134 in Q128 ES cells resulted in the generation of thousands of independent mutants, each one with different over-activated genes. Mutants that acquired resistance were expanded and further characterised, allowing the identification of genes as novel suppressors of mutant HTT toxicity.

b, Number of surviving cells scored by quantification of Crystal Violet staining-positive colonies upon treatment for 48 hours with the selected compounds. BafilomycinA and Rotenone had no effect on Q128 cells at doses affecting survival of Q15 cells, while MG132 and Tamoxifen further reduced survival of Q128 cells. Bars indicate the mean±SEM of at least 2 independent experiments. Data were normalized to Q15 vehicles samples. c, Left panel: representative images of parental ES cells electroporated with PB vector encoding for GFP (parental ES_GFP on the top), Q128 line electroporated with PB_GFP (Q128_GFP in the middle) and Q128 cells electroporated with pGG134 vector (Q128_pGG134 on the bottom) treated with MG132 for 5 days and stained with Crystal Violet. Rare Q128_pGG134 cells survived to MG132 treatment. Right panel: intensity of CV signal of surviving Q128_pGG134 colonies resulted significantly increased compared to Q128_GFP after mutagenesis and selection in presence of MG132 (left) or Tamoxifen (right). Bars indicate the mean±SEM of 4 independent experiments shown as dots. Each experiment has been normalized to PB_GFP vector. p-values were calculated with Two-tailed one sample Mann-Whitney U test.

d, Identification of integration sites in resistant clones. The gel electrophoresis on the left panel shows

Sp-PCR products from mutant clones MG15, MG16 and MG17. Single band corresponding amplification of 5′ end (red dashed square) of the PB vector in MG15, was excised from gel, purified and sequenced. The sequence obtained (top, right panel) includes a portion of genomic DNA followed by BstYI restriction site (GATC sequence) and the adapter sequence. Genomic sequence was then aligned to the mouse genome, allowing identification of the precise site of integration in each mutant cell line. Here, PB vector was found inserted upstream of the Kdm5b gene (bottom panel). e, Expression analysis by qPCR of Synj2, Kdm5b, Mtf1, Fbxo34 and Arid1b genes confirmed upregulated expression of such candidate targets in the corresponding clones, compared to both the parental and the Q128 cell line. Bars indicate the mean±SEM of 3 technical replicates shown as dots. Expression was normalised to the highest value.

f, Representative Crystal Violet staining images of Q128 cells and 5 clones selected from Q128_pGG134 mutant population (left panel). All clones were resistant to exogenous stressors while most of Q128 cells died and parental ES cells survived.

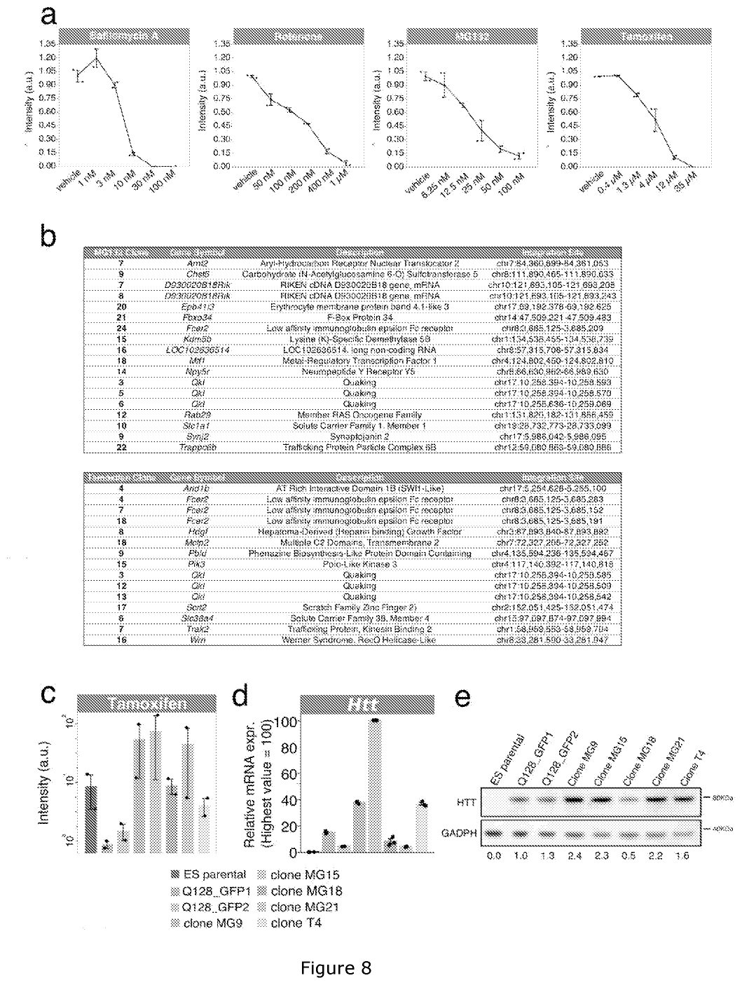

g, Intensity of Crystal violet signal in all 5 clones in analysis after 48 hrs of treatments with MG132 or Tamoxifen (MG132 shown, Tamoxifen in FIG. 8c). Data were normalized to Q128_GFP1. Bars indicate the mean±SEM of 2 independent experiments shown as dots.

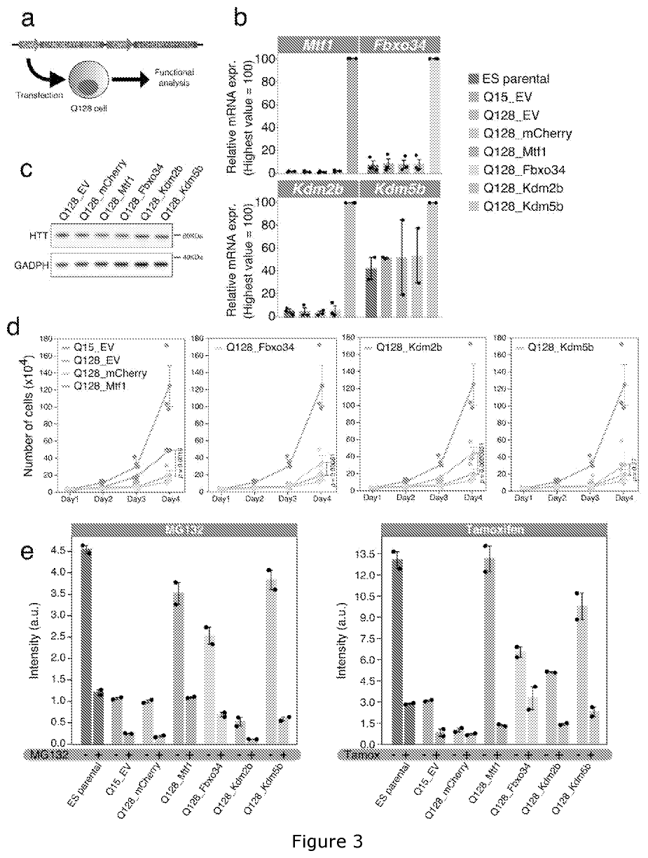

FIG. 3—Secondary validation of mutant HTT suppressors. a, Schematic representation of secondary validation experiment performed by stable expression in Q128 cells of a vector harboring cDNA of candidate genes under the control of a constitutive CAG promoter. An empty vector (EV) and a vector containing mCherry cDNA served as negative controls. b, Gene expression analysis by qPCR of Mtf1, Kdm2b, Kdm5b and Fbxo34 confirmed increased levels of genes in corresponding cell lines in which they were overexpressed. Bars indicate the mean±SEM of 3 independent experiments (Mtf1, Fbxo34, Kdm2b) and 2 independent experiments (Kdm5b) shown as dots. Expression was normalised to the highest value. c, Western Blot analyses showed that mutant HTT protein was present at comparable levels in Q128 cells transfected with different constructs. GAPDH was used as loading control. Uncropped gels are provided in the Source data file. d, Proliferation assay of the indicated cell lines. Bars indicate the mean±SEM of 3 independent experiments, shown as dots. p-values were calculated with Two-way Repeated Measure ANOVA, comparing each candidate to the Q128_EV sample.

e, Crystal violet quantification showing the number of surviving colonies in 128_Mtf1, Q128_Kdm5b and Q128_Fbxo34 cells after 48 hrs of treatments with MG132 (left panel) or Tamoxifen (right panel), compared to both the parental and the Q128 cell lines. Mtf1 significantly improved cell resistance to mutant HTT, while mild effects were observed upon overexpression of other candidates. Bars indicate the mean values from 2 technical replicates of a single experiment reported in the Source Data file (the same trend has been observed in 2 additional experiments).

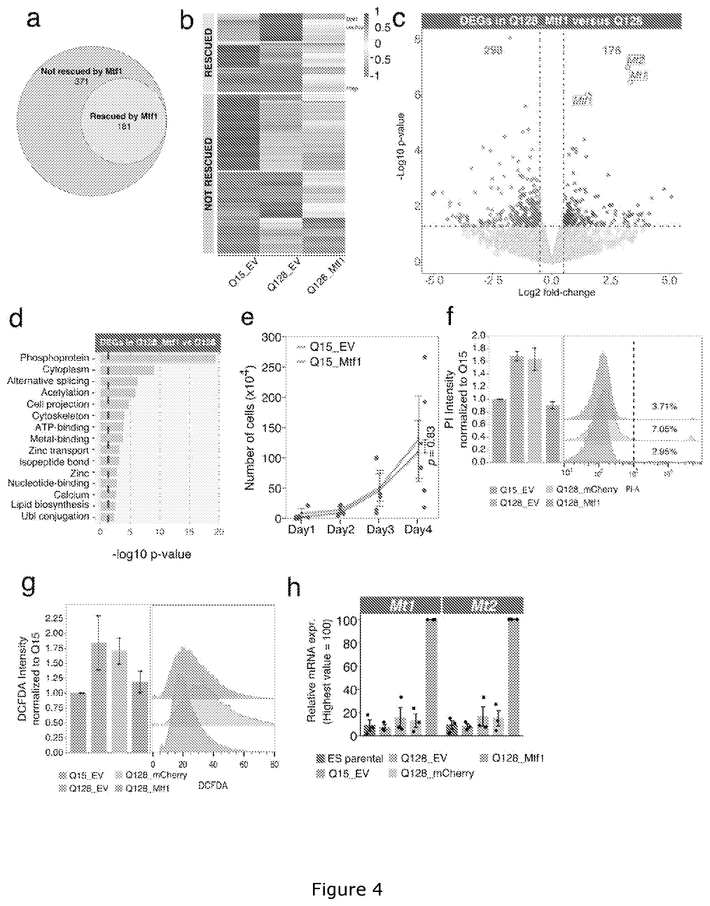

FIG. 4—Mtf1 regulates HD-related genes. a, Differential expressed genes rescued by Mtf1. Out of 552 DEGs between Q15 and Q128 (FIG. 1f), 181 genes (pink) were rescued by Mtf1 overexpression (p-value >=0.05 between Q128_Mtf1 and Q15_EV and log 2FC>|0.5| between Q128_Mtf1 and Q128_EV). b, Heatmap showing the rescue effect of Mtf1 overexpression on 552 DEGs between Q128 and Q15 cells. Z-score calculated on row-scaled expression values (Counts Per Million) are shown for each cell line (Q15, Q128 and Q128_Mtf1). Red and blue indicate high and low expression respectively. HD related genes Dpf1, Prnp, and Ube2cbp are highlighted. c, Transcriptome analysis of Q128_Mtf1 compared to Q128 cells. DOWN-regulated (Log 2 fold-change <0.5 and p-value <0.05) and UP-regulated (Log 2 fold-change >0.5 and p-value <0.05) genes are indicated in blue and red, respectively. Mt1 and Mt2, together with Mtf1 itself, are highlighted as the most significantly upregulated genes. Thresholds used are indicated by dashed lines. P-values were calculated with Wald Test with no adjustment. d, Gene list enrichment analysis for the transcriptional response induced by Mtf1 overexpression in Q128 cells. Black dashed line equals to p-value=0.05. p-values were calculated by Fisher Exact test using DAVID database. e, Proliferation assay of Q15 cells transfected with an empty vector (Q15_EV) or with a Mtf1 (Q15_Mtf1) encoding plasmid. No significant changes on cell proliferation rate upon Mtf1 overexpression was observed. Bars indicate the mean±SEM of 3 independent experiments shown as dots. p-values were calculated with Two-way Repeated Measure ANOVA. f, Measurement of cell death by Propidium Iodide uptake and Flow Cytometry. The staining revealed that Q128_Mtf1 (green) overexpression reduced cell death, compared to Q128 (orange) cells. The fraction of PI-positive cells (dashed line) was calculated for each sample and fold-changes were calculated relative to the Q15 samples. Bars indicate the mean±SEM of 2 independent experiments shown as dots. Representative flow cytometry plots are shown on the right. g, Measurement of 2′,7′-dichlorofluorescin diacetate (DCFDA) median fluorescence as an evaluation of Reactive Oxygen Species production. The overexpression of 128_Mtf1 (green) was able to reduce ROS production in cells expressing mutant HTT. Bars indicate the mean±SEM of 2 independent experiments shown as dots. Fold-changes were calculated relative to the Q15 samples. Representative flow cytometry profiles of ROS detection are represented in the right panel. Source data are provided as a Source Data file. h, Gene expression analysis by qPCR confirmed a strong upregulation of Mt1 and Mt2 genes in Q128_Mtf1 cells (green). Bars indicate the mean of 3 independent experiments shown as dots. Expression was normalised to the highest value.

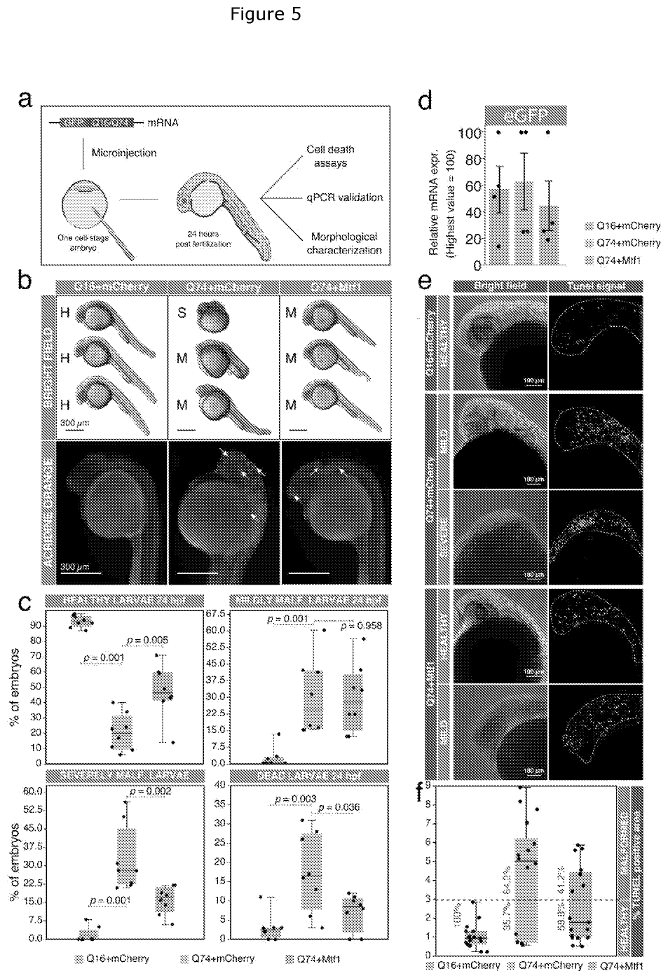

FIG. 5—Mtf1 counteracts mutant HTT effects in zebrafish. a, Exon 1 the huntingtin (HTT) coding sequence, including either 16 or 74 CAG repeats were cloned into pCS2 plasmids, fused in frame with the eGFP coding sequence. After in vitro transcription, Q16eGFP and Q74eGFP mRNAs were injected into the yolk of one cell-stage embryos. 24 hours post fertilization (hpf), embryos were collected for phenotypic and molecular analyses. b, Representative images of 24 hours-stage embryos, injected with either Q16eGFP+mCherry, Q74eGFP+mCherry or with Q74eGFP+Mtf1 (250 pg/embryo+250 pg/embryo), stained with the Acridine Orange. Top: Bright-field images show that the morphology of embryos was affected by Q74eGFP+mCherry mRNA injection. Bottom: fluorescent microscopy shows increased Acridine Orange positive foci (white arrows) upon Q74eGFP+mCherry injection. Images are lateral views with anterior at the top. c, Percentage of dead, malformed (severe or mild) and healthy embryos counted 24 hpf in 8 independent injection experiments, shown as dots. A total of 407, 511 and 621 embryos were analysed for Q16+mCherry, Q74+mCherry and Q74+Mtf1 samples, respectively. Box plots indicate 1st, 2nd and 3rd quartile; whiskers indicate minimum and maximum. P-values were calculated with the Unpaired two-tailed Mann-Whitney U test. d, eGFP gene-expression analysis by qPCR of Zebrafish embryos microinjected with eGFP and Q74+Mtf1 mRNAs. Bars indicate the mean±SEM of 4 independent experiments shown as dots. Expression was normalised to the highest value. e, Representative images of TUNEL assay on 30 hours-stage embryos from two independent experiments, injected with either Q16+mCherry or with Q74+mCherry or with Q74+Mtf1. Multiple focal planes were scanned for each embryo, spanning the entire depth of anterior structures, and z-projections were obtained on either bright-field and fluorescence channels. Q16+mCherry injected embryos revealed some basal TUNEL positivity, due to physiological apoptotic-dependent remodelling occurring at this stage of development. f, Quantification of the percentage of TUNEL positive area over the total area (excluding the yolk region). Each dot represents an embryo (Q16+mCherry=15, Q74+mCherry=14, Q74+Mtf1=17). Box plots indicate 1st, 2nd and 3rd quartile; whiskers indicate minimum and maximum. The percentages of malformed and healthy embryos are shown in red and green, respectively. Healthy embryos are characterized by reduced TUNEL signal.

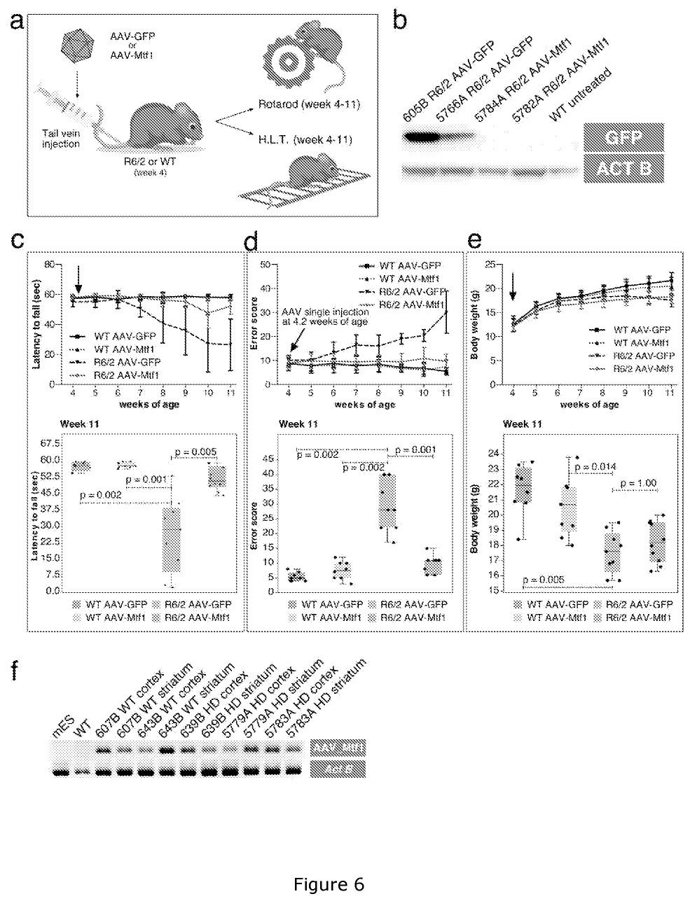

FIG. 6—AAV-vector delivery of Mtf1 alleviates motor deficit in R6/2 mice. a, Schematic representation of experimental strategy for motor behaviour tests in HD mouse model injected with AAVs packaged with Mtf1 or GFP. Tail-vein injections were performed at 4 weeks of age. Motor performance was assessed by Horizontal Ladder Task and Rotarod tests. b, Western Blot of GFP confirmed viral expression in brain lysate of four week old R6/2 mice tail-vein injected with AAV-containing GFP and therefore the ability of AAVs to cross the brain blood barrier. AAV-Mtf1 injected mice were used as negative control. Act B was used as loading control.

c, Injection of AAV-Mtf1 ameliorates motor function in R6/2 mice (red line). Motor performance assessed by Rotarod test considers the time of latency to fall: R6/2 mice injected with AAV-GFP vector fall more rapidly than wild-type littermates and R6/2 mice injected with AAV-Mtf1. Arrows indicate when treatment started. Top: line plots show mean+/−standard deviation of each experimental group at each time point. Bottom: boxplots of the indicated experimental groups at 11 weeks of age. Box plots indicate 1st, 2nd and 3rd quartile; whiskers indicate minimum and maximum. For each group, the number of injected mice is shown as dots (WT AAV-GFP=8; WT AAV-Mtf1=9; R6/2 AAV-GFP=9; R6/2 AAV-Mtf1=10). P-values were calculated with the Unpaired two-tailed Mann-Whitney U test with Bonferroni correction. d, Analysis of motor coordination on Horizontal Ladder task. R6/2 mice injected with AAV-Mtf1 (red line) totalized a lower error score compared to R6/2 mice injected with AAV-GFP vector (black dashed line), especially in the late weeks of age, when symptoms appear. Arrows indicate when treatment started. Box plots indicate 1st, 2nd and 3rd quartile; whiskers indicate minimum and maximum. For each group, the number of injected mice is shown as dots (WT AAV-GFP=8; WT AAV-Mtf1=8; R6/2 AAV-GFP=9; R6/2 AAV-Mtf1=10). P-values were calculated with the Unpaired two-tailed Mann-Whitney U test with Bonferroni correction. e, Average body weight of R6/2 and WT mice after viral injection with either AAV-GFP or AAV-Mtf1. Box plots indicate 1st, 2nd and 3rd quartile; whiskers indicate minimum and maximum. For each group, the number of injected mice is shown as dots (WT AAV-GFP=8; WT AAV-Mtf1=9; R6/2 AAV-GFP=9; R6/2 AAV-Mtf1=10). P-values were calculated with the Unpaired two-tailed Mann-Whitney U test with Bonferroni correction. f, PCR on total DNA confirmed effective in vivo brain delivery of Mtf1 both in brain cortex and striatum tissues. Act B was used as a positive control for PCR reaction.

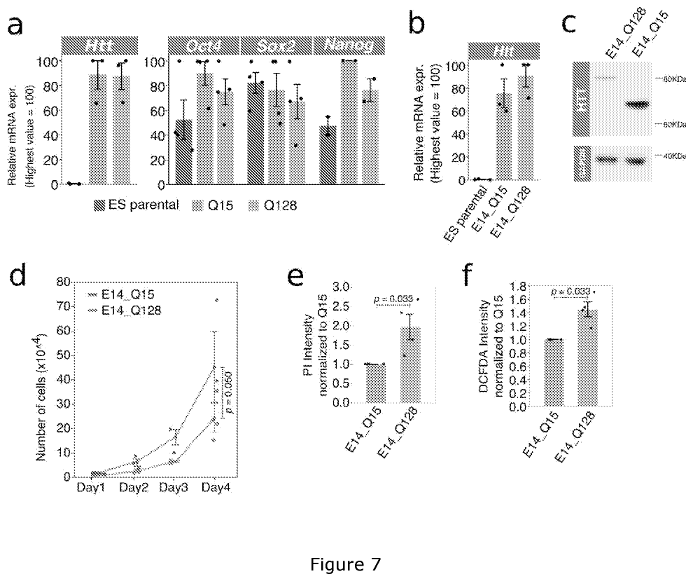

FIG. 7—Establishment and characterization of mutant HTT-expressing ES cells. a, Gene expression analysis by qPCR of HTT gene (on left) and pluripotency markers Oct4, Sox2 and Nanog (right panels) in Q15 (blue) and Q128 (orange) cells, compared to parental ES line (Rex1GFP-d2). Bars indicate the mean±SEM (standard error of the mean) of 2 (for Nanog), 3 (for HTT) or 4 (for Oct4 and Sox2) independent experiments shown as dots. Results showed similar elevated expression levels of HTT mRNA for both HD lines and contextually the retainment of pluripotency features. Expression was normalised to the highest value. b, Gene expression analysis by qPCR of HTT gene in wild-type mouse ES cells (E14TG2a) expressing either Q15 (blue) or Q128 (orange) constructs, compared to parental ES line. Bars indicate the mean±SEM of 3 independent experiments shown as dots. Results showed similar elevated expression levels of HTT mRNA for both HD lines. Expression was normalised to the highest value. c, Western Blot of HTT confirmed the correct production of a 80 kDa and a 65 kDa form of HTT protein in E14_Q128 and E14_Q15 cells respectively. GAPDH was used as loading control. d, Proliferation assay of E14_Q128 (orange) and E14_Q15 (blue) ES cells showed pronounced impairment in cell proliferation due to mutant HTT expression. Bars indicate the mean±SEM of 3 independent experiments, shown as dots. p-values were calculated with Two-way Repeated Measure ANOVA. e, Measurement of cell death by Propidium Iodide uptake and Flow Cytometry. E14_Q128 cells (orange) display higher cell death, compared to E14_Q15 (blue) cells. Bars indicate the mean±SEM of 4 independent experiments shown as dots. Fold-changes were calculated relative to the Q15 samples. p-values were calculated with One-tailed one sample Mann-Whitney U test. Source data are provided as a Source Data file. f, Measurement of 2′,7′-dichlorofluorescin diacetate (DCFDA) fluorescence as an evaluation of Reactive Oxygen Species production in E14_Q15 (blue) versus E14_Q128 (orange) cells. Bars indicate the mean±SEM of four independent experiments shown as dots. Fold-changes were calculated relative to the Q15 samples. p-values were calculated with One-tailed one sample Mann-Whitney U test.

FIG. 8-A gain-of-function screen for suppressors of mutant HTT toxicity. a, Number of surviving parental ES cells quantified by Crystal Violet staining upon treatment for 48 hours with the indicated compounds. Bars indicate the mean±SEM of 2 independent experiments. Data were normalized to cells treated with vehicles. b, List of target genes identified by Sp-PCR and analysis of PCR bands for all MG or Tamoxifen clones collected. For 9 clones (out of 44 collected) we could not identify the site of integration for technical limitations, such as multiple integration sites or failure to detect any signal. c, Crystal Violet quantification showing the number of surviving colonies in all 5 clones in analysis after 48 hrs of treatments with Tamoxifen. Data were normalized to Q128 control. Bars indicate the mean±SEM of 2 independent experiments shown as dots. d, qPCR analysis showed similar high expression levels of HTT mRNA in all clones, as well as in Q128 Htt ES cells, confirming that Q128 mRNA was not silenced during the screening procedure. Bars indicate the mean±SEM of 3 technical replicates shown as dots. Expression was normalised to the highest value.

e, Western Blot confirmed that HTT protein was still present in all clones. GAPDH was used as loading control. Values shown below each clone are the mean (3 technical replicates) of HTT intensity normalised to GAPDH intensity.

FIG. 9—Network analysis of candidate suppressors of mutant HTT toxicity. a, Gene list enrichment analysis for the genes identified from NGS screenings, revealed a statistically significant enrichment for categories linked to proteasome degradation (e.g. ubiquitin conjugation, p-value=9.77E-04) or vesicular trafficking (e.g. acetylation, p-value=0.005) and transcriptional regulators (e.g. methylation, p-value=0.001). P-values were calculated by Fisher Exact test using DAVID database.

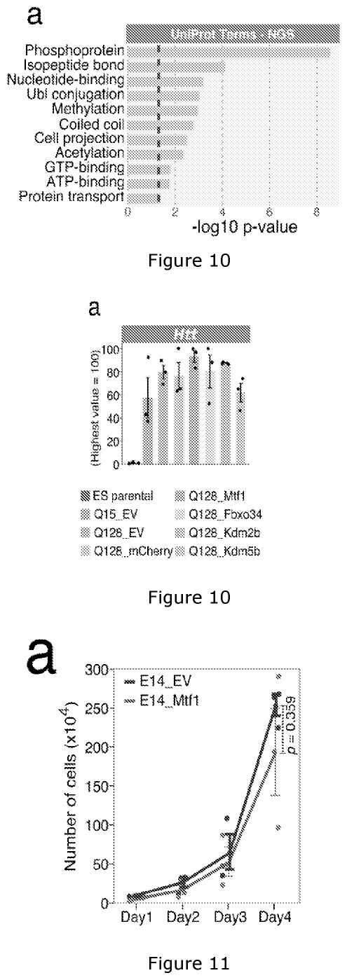

FIG. 10—Secondary validation of mutant HTT suppressors. a, qPCR analyses for HTT mRNA showed that HTT expression was not affected by co-expression of candidate genes. Bars indicate the mean±SEM of 3 independent experiments shown as dots. Expression was normalised to the highest value.

FIG. 11—Mtf1 regulates HD-related genes. a, Proliferation assay of E14 cells transfected with an empty vector (E14_EV) or with a Mtf1 encoding plasmid (E14_Mtf1). No significant changes on cell proliferation rate upon Mtf1 overexpression was observed. Bars indicate the mean±SEM of 3 independent experiments shown as dots. p-values were calculated with Two-way Repeated Measure ANOVA.

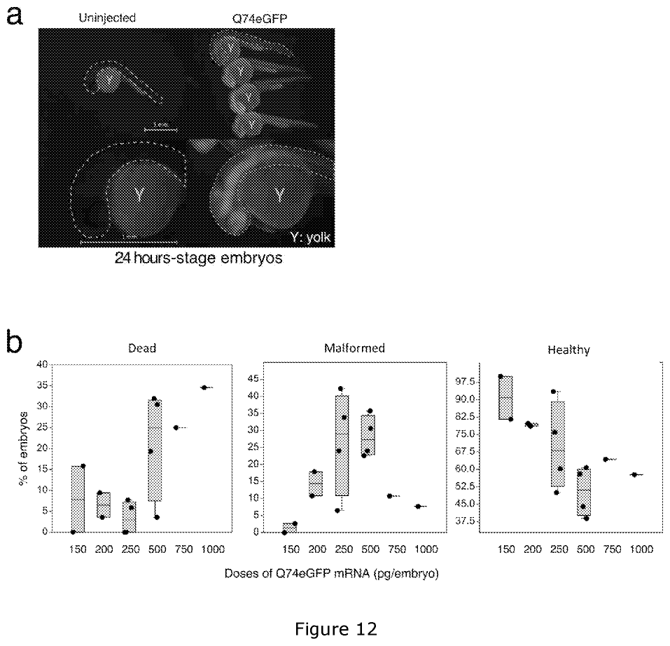

FIG. 12—Mtf1 counteracts mutant HTT effects in zebrafish. a, Representative images obtained by fluorescent microscopy of 24 hours-stage embryos injected with Q74eGFP mRNA (250 pg/embryo, right panels) compared to uninjected controls (left panels). Dashed lines represent the region of interest, while the yolk (Y) shows autofluorescence. Q74eGFP mRNA-injected embryos exhibited a fluorescent signal along the entire embryo. b, Percentages of dead, malformed and healthy embryos phenotypically scored 24 hpf and obtained after injecting increasing doses of Q74eGFP mRNA, ranging from 150 to 1000 pg/embryo. Doses above 500 pg/embryo were highly toxic for embryos, leading many fish to die, while doses below 500 pg/embryo were more tolerated. The dose of 250 pg/embryo showed the highest rate of malformations with the lowest level of death, and for this reason it was chosen for the following experiments. Each dot represents an independent experiment.

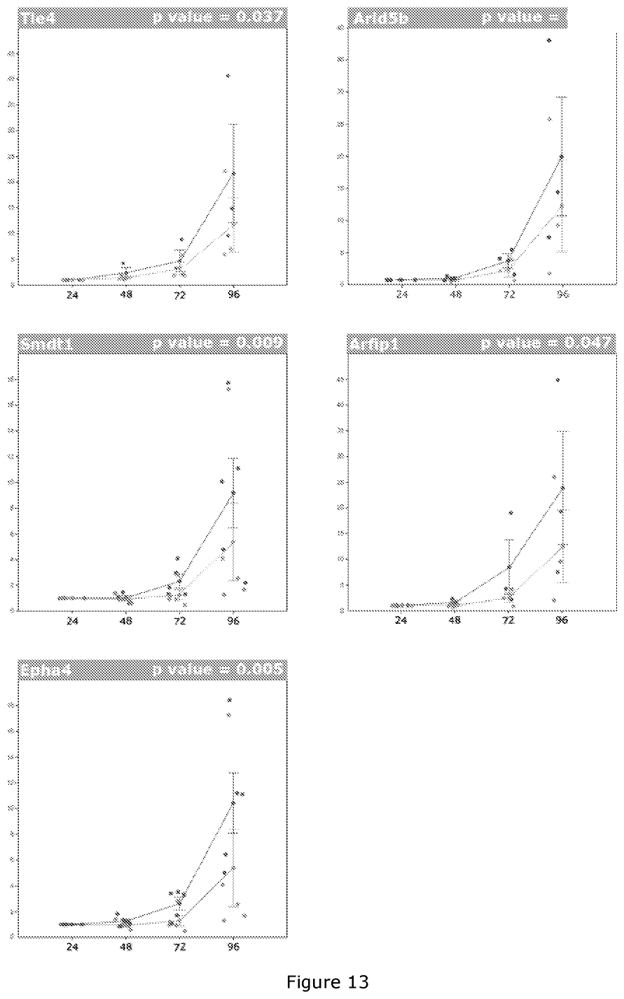

FIG. 13 Proliferation assay of Q128 cells transfected with an empty vector (Q128_EV) or with candidate therapeutic gene encoding plasmids: Arid5b (Q128_Arid5b), Smdt1 (Q128_Smdt1), Epha4 (Q128_Epha4), Tle4 (Q128_Tle4), Arfip1 (Q128_Arfip1).

Cell proliferation was assessed by plating 30,000 ES cells in a 12-well plate in presence of Puromycin 6 μg/ml. Cells were counted every 24 h for 4 days.

Significant rescue on cell proliferation rate upon every candidate gene's overexpression was observed. Bars indicate the mean±SEM of 3 independent experiments for Arid5b, Tle4, Arfip1, and 5 independent experiments for Smdt1, Epha4, shown as dots. p-values were calculated with Two-way Repeated Measure ANOVA, comparing each candidate to the Q128_EV sample.

### DETAILED DESCRIPTION OF THE INVENTION

The authors of the present invention were able to identify a number of therapeutic proteins that, when recombinantly expressed or when introduced in cells that are model cells of a polyQ disease or administered to model animals of a polyQ disease (i.e. cells or animals expressing mutated proteins causing polyQ diseases, e.g. as depicted in Table 1), are capable of reducing the toxicity of the mutated protein causing said polyQ disease thereby providing a new tool for the treatment of said diseases.

The present invention therefore relates to a therapeutic protein capable of reducing the toxicity of mutated proteins causing a polyQ disease, for use in the treatment of said diseases, by way of example by protein or gene therapy.

In other words, the invention relates to a therapeutic protein, i.e. a protein, whose expression or delivery in the cells/tissue/organism of interest, reduces the toxicity elicited by a polyQ mutated protein responsible of a polyQ disease, i.e. a pathogenic polyQ mutated protein resulting from the expression of a mutated gene with an anomalous increase of CAG triplets with respect to the wild type gene.

In an embodiment of the invention, the pathogenic mutated protein causing a polyQ disease is expressed by one or more of ATN1, HTT, AR, ATXN1, ATXN2, ATXN3, CACNA1A, ATXN7, PPP2R2B, TBP mutated genes, the mutation being the anomalous expansion of a CAG triplet within a CAG repeat, wherein said mutated protein comprises a pathological number of polyQ residues.

PolyQ diseases are well-known in the art; Table 1 below provides a list of genes in which a mutation consisting of an expansion of a CAG triplet within a CAG repeat region results in the expression of mutated proteins comprising an anomalous repeat of Q residues, herein also defined as PolyQ repeats, said proteins causing the so-called polyQ diseases.

| | | | Normal | Pathogenic |
|---|---|---|---|---|
| | | | PolyQ | PolyQ |
| Type | Gene | PROTEIN | repeats | repeats |
| DRPLA (Dentatorubropallidoluysian | ATN1 or | atrophin-1 | 6-35 | 49-88 |
| atrophy) | DRPLA | | | |
| HD (Huntington's disease) | HTT | huntingtin | 6-35 | 36-250 |
| SBMA (Spinal and bulbar muscular | AR | androgen | 4-34 | 35-72 |
| atrophy)^{[3]} | | receptor | | |
| SCA1 (Spinocerebellar ataxia Type | ATXN1 | ataxin-1 | 6-35 | 49-88 |
| 1) | | | | |
| SCA2 (Spinocerebellar ataxia Type | ATXN2 | ataxin-2 | 14-32 | 33-77 |
| 2) | | | | |
| SCA3 (Spinocerebellar ataxia Type | ATXN3 | ataxin-3 | 12-40 | 55-86 |
| 3 or Machado-Joseph disease) | | | | |
| SCA6 (Spinocerebellar ataxia Type | CACNA1A | voltage- | 4-18 | 21-30 |
| 6) | | dependent P/Q- | | |
| | | type calcium | | |
| | | channel subunit | | |
| | | alpha-1A | | |
| SCA7 (Spinocerebellar ataxia Type | ATXN7 | ataxin-7 | 7-17 | 38-120 |
| 7) | | | | |
| SCA12 (Spinocerebellar ataxia Type | PPP2R2B | serine/threonine- | 7-41 | 43-51 |
| 12)^{[4]} | | protein | | |
| | | phosphatase 2A | | |
| | | 55 kDa | | |
| | | regulatory | | |
| | | subunit B beta | | |
| | | isoform | | |
| SCA17 (Spinocerebellar ataxia Type | TBP | TATA-box | 25-42 | 47-63 |
| 17) | | binding protein | | |

Therefore, according to the invention, said polyQ disease is one of Dentatorubropallidoluysian atrophy, Huntington's disease, Spinal and bulbar muscular atrophy, Spinocerebellar ataxia Type 1, Spinocerebellar ataxia Type 2, Spinocerebellar ataxia Type 3, Spinocerebellar ataxia Type 6, Spinocerebellar ataxia Type 7, Spinocerebellar ataxia Type 12 or Spinocerebellar ataxia Type 17.

In a preferred embodiment the protein causing the polyQ disease is expressed by the HTT gene and the related polyQ disease is Huntington's corea alias Huntington's disease.

According to the invention, the therapeutic protein for use for the treatment of a polyQ disease, capable of reducing the toxicity of a mutated protein causing a polyQ disease is one of: metal regulatory transcription factor 1, lysine-specific demethylase 5B, lysine-specific demethylase 2B, F-box only protein 34, essential MCU regulator, mitochondrial, Ephrin type-A receptor 4, Transducin-like enhancer protein 4, Arfaptin-1, AT-rich interactive domain-containing protein 5B or isoforms thereof, or homologs of said therapeutic proteins having a sequence homology, defined as at least 55% coverage and at least 56% identity with one of said therapeutic protein; wherein said isoforms and homologs proteins retain the capability of the therapeutic protein of which they are isoforms or homologs as defined herein, of reducing the toxicity of the mutated protein causing a given polyQ disease.

Table 2 below summarises the names of the human and mouse genes coding for said therapeutic proteins as well as the name of the therapeutic protein coded by each gene.

Each gene listed below is well characterised in the art, both gene sequence and cDNA sequence, is associated to an NBCI gene ID and the coded protein sequence is available in public protein databases. Therefore, reference to the gene, name, to the gene NCBI ID as well to the protein name provides the skilled person with all the necessary information on the nucleotide sequences coding for the therapeutic proteins of interest of the invention as well as on the aminoacidic sequence of each of said therapeutic proteins.

TABLE 2

| OFFICIAL | OFFICIAL | | | |
|---|---|---|---|---|
| GENE | GENE | | | |
| SYMBOL | SYMBOL | OFFICIAL GENE NAME | | NBCI Gene |
| Mouse | Human | (PROTEIN NAME) | ALIASES | ID |
| Mtf1 | MTF1 | metal regulatory | ZRF; MTF-1 | 4520 |
| | | transcription factor 1 | | |
| | | (metal regulatory | | |
| | | transcription factor 1) | | |
| Kdm5b | KDM5B | lysine demethylase 5B | CT31; PLU1; PUT1; | 10765 |
| | | (lysine-specific | MRT65; PLU-1; | |
| | | demethylase 5B) | JARID1B; | |
| | | | PPP1R98; RBP2- | |
| | | | H1; RBBP2H1A | |
| Kdm2b | KDM2B | lysine demethylase 2B | CXXC2; Fbl10; | 84678 |
| | | (lysine-specific | PCCX2; FBXL10; | |
| | | demethylase 2B) | JHDM1B | |
| Fbxo34 | FBXO34 | F-box protein 34 | Fbx34; CGI-301 | 55030 |
| | | (F-box only protein 34) | | |
| Smdt1 | SMDT1 | single-pass membrane | DDDD; EMRE; | 91689 |
| | | protein with aspartate | C22orf32 | |
| | | rich tail 1 | | |
| | | (essential MCU | | |
| | | regulator, mitochondrial) | | |
| Epha4 | EPHA4 | EPH receptor A4 | EK8; SEK; HEK8; | 2043 |
| | | (ephrin type-A receptor | TYRO1 | |
| | | 4) | | |
| Tle4 | TLE4 | TLE family member 4, | ESG; BCE1; ESG4; | 7091 |
| | | transcriptional | GRG4; BCE-1; Grg- | |
| | | corepressor | 4; E(spl); E(spl) | |
| | | (transducin-like enhancer | | |
| | | protein 4) | | |
| Arfip1 | ARFIP1 | ADP ribosylation factor | HSU52521 | 27236 |
| | | interacting protein 1 | | |
| | | (arfaptin-1) | | |
| Arid5b | ARID5B | AT-rich interaction | MRF2; DESRT; | 84159 |
| | | domain 5B | MRF-2 | |
| | | (AT-rich interactive | | |
| | | domain-containing | | |
| | | protein 5B) | | |

The therapeutic proteins identified by the inventors are suitable for use in the treatment of polyQ diseases.

Isoforms of said therapeutic proteins, retaining the capability of reducing the toxicity of a mutated protein causing a polyQ disease and nucleotide sequences coding the same, for use in the treatment of said disease are also encompassed by the present invention.

An example of suitable isoforms according to the invention, indicated by NM or XM or NR NCBI annotation number is provided in Table 3 below:

TABLE 3

| Therapeutic protein | |
|---|---|
| from table 2 | Isoforms |
| MTF1 | NM_005955.3; XM_011541494.1; XM_011541491.2; XM_011541493.3 |
| KDM5B | NM_001314042.1; NM_001347591.1; NM_006618.5 |
| KDM2B | NM_001005366.2; NM_032590.5; XM_011538867.3; XM_011538868.3; |
| | XM_005253955.4; XM_005253956.4; XM_005253961.5; XM_011538875.3; |
| | XM_011538869.2 |
| FBX034 | NM_017943.4; NM_152231.2; XM_006720185.3; XM_017021391.1; |
| | XM_017021392.1; XM_017021393.2; XM_005267791.4 |
| SMDT1 | NM_033318.5; NR_146715.2; XM_011530509.3; NR_146717.1 |
| EPHA4 | NM_001304536.2; NM_001304537.2; NM_001363748.2; NM_004438.5 |
| TLE4 | NM_001282748.2; NM_001282749.2; NM_001282753.2; NM_001282760.2; |
| | NM_001351541.2; NM_001351542.2; NM_001351543.2; NM_001351546.2; |
| | NM_001351547.2; NM_001351550.2; NM_001351552.2; NM_001351556.2; |
| | NM_001351558.2; NM_001351560.2; NM_001351562.2; NM_001351563.2; |
| | NM_001351564.2; NM_007005.6; NR_104239.2; XM_011518952.1; |
| | XM_006717264.1; XM_011518958.1; XM_006717268.1; XM_011518967.1; |
| | XM_011518969.1; XM_011518956.1; XM_011518957.1; XM_011518959.1; |
| | XM_011518965.1; XM_017015081.1; XM_011518960.1; XM_011518966.1; |
| | XM_017015083.1; XM_011518963.1; XM_011518964.1; XM_011518955.1; |
| | XM_011518961.1; XM_011518962.1; XM_011518968.1; XM_011518954.1; |
| | XM_017015068.1; XM_017015075.1; XM_011518970.3; XM_017015070.1; |
| | XM_017015067.1; XM_011518953.2; XM_017015069.2; XM_011518972.3; |
| | XM_024447659.1 |
| ARFIP1 | NM_001025593.3; NM_001025595.3; NM_001287431.2; NM_001287432.2; |
| | NM_001287433.2; NM_014447.4 |
| ARID5B | NM_001244638.2; NM_032199.3 |

Homologs of said therapeutic proteins having a sequence homology, defined as at least 55% coverage and at least 49% identity with one of said therapeutic proteins, retaining the capability of reducing the toxicity of a mutated protein causing a polyQ disease and nucleotide sequences coding the same, for use in the treatment of said disease are also encompassed by the present invention.

In a preferred embodiment the homolog of said therapeutic has a sequence homology defined as at least 88% coverage and at least 49% identity with one of said therapeutic proteins, or defined as at least 55% coverage and at least 58% identity with one of said therapeutic proteins, or as at least 95% coverage and at least 55% identity with one of said therapeutic proteins, or as at least 95% coverage and at least 64% identity with one of said therapeutic proteins, or as at least 97% coverage and at least 64% identity with one of said therapeutic proteins, or as at least 99% coverage and at least 64% identity with one of said therapeutic proteins, as at least 99% coverage and at least 70% identity with one of said therapeutic proteins, or as at least 99% coverage and at least 77% identity with one of said therapeutic proteins, or as at least 99% coverage and at least 79% identity with one of said therapeutic proteins, and retains the capability of reducing the toxicity of a mutated protein causing a polyQ disease and nucleotide sequences coding the same, for use in the treatment of said disease are also encompassed by the present invention.

An example of suitable homologs according to the, is provided in Table 4 below:

TABLE 4

| Therapeutic | reference | | | | |
|---|---|---|---|---|---|
| protein | NCBI | | | | |
| from | gene | | | Coverage | Identity |
| Table 2 | ID | Homolog | REFERENCE NCBI | % | % |
| KDM5B | 10765 | KDM5A | 5927 | 94 | 56.35 |
| | | KDM5C isoform 3 | NP_001269551.1 | 88 | 49.05 |
| KDM2B | 84678 | FBXL11 | 22992 | 55 | 58.61 |
| EPHA4 | 2043 | EPHA5 isoform d and | NP_001268695.1 | 99 | 65.99 |
| | | b | NP_872272.2 | | |
| | | EPHA5 isoform a and c | NP_004430.4 | 99 | 64.41 |
| | | | NP_001268694.1 | | |
| | | EPHA7 isoform 2 | NP_001275558.1 | 97 | 64.08 |
| | | precursor | | | |
| | | EPHA7 isoform 5 | NP_001363395.1 | 97 | 64.17 |
| | | precursor | | | |
| | | EPHA7 isoform 1 | NP_004431.1 | 97 | 64.2 |
| | | precursor | | | |
| | | EPHA7 isoform 4 | NP_001363394.1 | 97 | 64.29 |
| | | precursor | | | |
| | | EPHA5 isoform f | NP_001305690.1 | 95 | 64.99 |
| | | EPHA5 isoform e | NP_001268696.1 | 95 | 65.12 |
| | | EPHA3 isoform a | NP_005224.2 | 96 | 64.33 |
| | | precursor | | | |
| | | EPHA3 isoform X1 | XP_005264772.1 | 96 | 64.44 |
| TLE4 | 7091 | TLE3 isoform e | NP_001269908.1 | 99 | 79.8 |
| | | TLE3 isoform X5 | XP_005254680.1 | 99 | 78.82 |
| | | TLE3 isoform d | NP_001269909.1 | 99 | 79.5 |
| | | TLE3 isoform b | NP_001098662.1 | 99 | 79.31 |
| | | TLE3 isoform X7 | XP_011520282.1 | 99 | 79.11 |
| | | TLE3 isoform X4 | XP_011520280.1 | 99 | 78.53 |
| | | TLE3 isoform X3 | XP_005254679.1 | 99 | 78.34 |
| | | TLE3 isoform a | NP_005069.2 | 99 | 79.01 |
| | | TLE3 isoform X2 | XP_011520279.1 | 99 | 78.14 |
| | | TLE3 isoform X1 | XP_011520278.1 | 99 | 78.05 |
| | | TLE3 isoform f | NP_001269910.1 | 98 | 79.22 |
| | | TLE3 isoform X15 | XP_005254690.1 | 99 | 78.93 |
| | | TLE3 isoform X10 | XP_005254685.1 | 99 | 77.96 |
| | | TLE3 isoform c | NP_065959.1 | 99 | 78.44 |
| | | TLE3 isoform X8 | XP_005254682.1 | 99 | 77.48 |
| | | TLE1 isoform X12 | XP_006717323.1 | 99 | 71.45 |
| ARFIP1 | 27236 | ARFIP2 isoform 9 | NP_001363487.1 | 98 | 59.94 |
| | | ARFIP2 isoform 5 | NP_001357335.1 | 98 | 59.64 |
| | | ARFIP2 isoform 10 | NP_001363489.1 | 98 | 59.64 |
| | | ARFIP2 isoform 1 | NP_001229783.1 | 98 | 55.41 |
| | | ARFIP2 isoform 8 | NP_001357341.1 | 98 | 57.86 |
| | | ARFIP2 isoform 7 | NP_001357338.1 | 98 | 58.75 |

In an embodiment a therapeutic protein, or an isoform or homolog thereof preferably in the form of a recombinant protein, listed in Tables 2, 3 and 4 (or a combination thereof) is administered, through suitable delivery means, to a subject in need of a treatment for a polyQ disease wherein the polyQ disease is one of the ones listed in Table 1.

In a preferred embodiment, a therapeutic protein listed in Table 2 or an isoform or homolog thereof listed in tables 3 and 4 (or a combination thereof) is provided to a subject in need of a treatment for a polyQ disease through protein delivery or through a delivery system of nucleotide sequences that will be expressed in the host, by way of example through a gene therapy treatment. In a preferred embodiment, the therapeutic protein/s (and/or isoforms and/or homologs thereof) is/are expressed by the host following administration of a suitable expression vector or delivery system or vector suitable for gene therapy, or adeno-associated viral vector AAV or Lentiviral vector, or nanoparticle, or LNP or nucleotide construct, this embodiment is also defined in the present description generally as “gene therapy”.

Gene therapy is well known in the art. Preferably, according to the present invention, gene therapy is carried out by the administering to a subject in need of a polyQ disease treatment, a suitable expression vector or delivery system or vector suitable for gene therapy, or adeno-associated viral vector AAV or Lentiviral vector, or nanoparticle, or LNP or nucleotide sequences (such as, e.g. mRNA molecules), coding for a therapeutic protein of Table 2 and/or isoforms thereof as defined herein (e.g. table 3), and/or homologs thereof as defined herein (e.g. table 4) or a mixture thereof, directing the expression of the therapeutic protein of interest in the cell or in the host to which they have been administered.

The invention, therefore also relates to an expression vector or delivery system or a vector suitable for gene therapy, or an adeno-associated viral vector AAV or a Lentiviral vector, or a nanoparticle, or an LNP or a nucleotide sequence, coding for a therapeutic protein, isoform or homolog thereof as defined above, preferably selected from the therapeutic proteins, isoforms or homolog thereof of Tables 2-4; to said vector or delivery system or a vector suitable for gene therapy, or an adeno-associated viral vector AAV or a Lentiviral vector, or a nanoparticle, or an LNP or a nucleotide sequence for use in the treatment of a polyQ disease such as a disease of Table 1; to a pharmaceutical composition comprising said vector or delivery system or a vector suitable for gene therapy, or an adeno-associated viral vector AAV or a Lentiviral vector, or a nanoparticle, or an LNP or nucleotide sequence and at least a pharmaceutically acceptable carrier, and to a method of treatment of a polyQ disease comprising administering to a patient in need thereof a therapeutically effective amount of said vector or composition.

Expression vectors, in particular gene therapy vectors are known in the art. Preferred vectors are vectors that are capable to direct, upon administration, the synthesis of the aforementioned therapeutic protein, isoform or homolog thereof, in a subject affected by a polyQ disease, preferably in the tissues affected by said disease.

In an embodiment of the invention, the construct used for polyQ disease therapy according to the invention comprises a nucleotide sequence, e.g. a cDNA or an RNA coding for the therapeutic protein of interest or an isoform or a homolog thereof as defined above, preferably one of Tables 2-4, under the control of a promoter (i.e. operably linked to a promoter), such as a constitutive promoter, an inducible promoter, an ubiquitous promoter, a tissue specific promoter, preferably a constitutive promoter, thereby providing the expression of the desired therapeutic protein.

Preferably, said construct comprising said nucleotide sequence, e.g. a cDNA or RNA will be designed in order to provide the recombinant expression of a therapeutic protein of the same species to be treated, therefore, in a preferred embodiment, when the subject is a human subject, the cDNA of the human gene will be expressed resulting in the expression of the recombinant human therapeutic protein of interest, isoform or homolog thereof. In a preferred embodiment the nucleotide sequence of the invention may be designed in order to contain optimised codons.

In an embodiment of the invention, said therapeutic protein, isoform or homolog thereof is recombinantly expressed by an AAV vector.

Therefore, the invention also relates to a gene therapy expression adeno-associated viral vector, AAV, comprising a DNA sequence (cDNA) coding for a therapeutic protein, isoform or homolog thereof, capable of reducing the toxicity of mutated proteins causing polyQ diseases as defined above. A suitable example is provided in tables 2-4.

In an embodiment of the invention the vector is an AAV vector.

For polyQ diseases in which the CNS is affected, an AAV vector having an optimal capsid serotype for CNS gene therapy can be selected. The skilled person knows, by way of example, that optimal capsid serotypes for CNS gene therapy are AAV1, AAV2, AAV4, AAV5, AAV6, AAV7, AAV8, AAV9, AAV10, AAV11, AAV12, AAV13, AAV-PHP.B, AAV-PHP.eB, AAV-BR1, AAV-Retro, scAAV, AAVrh.8, AAVrh.10, AAV.miRNA AAV-DJ, AAV-LK03, AAV2.7m8.

Protocols for producing AAV vectors are known in the art, any of the known protocols can be used to carry out the invention.

In order to carry out gene therapy, the vector of the invention has to be delivered to the target cells.

Therefore, the invention also relates to a viral particle comprising a viral capsid and a nucleotide sequence coding for the therapeutic protein or isoform or homolog thereof according to any of the embodiments described above and claimed.

In a preferred embodiment, the viral particle is an AAV particle, therefore a particle consisting of a an AAV capsid and an adeno-associated viral vector (AAV) according to any of the embodiments defined above.

In one embodiment of the invention said vector has an optimal serotype for CNS gene therapy as previously described.

For AAV vectors, several AAV capsid proteins are known in the art, and several AAV capsids can be designed depending on the target cells for the therapy.

AAV is known to enter cells through interactions with carbohydrates present on the surface of the target cells, typically sialic acid, galactose and heparin sulphate. It is known that differences in sugar-binding preferences encoded in capsid sequence can influence cell-type transduction preferences of the various AAV.

AAV9 preferential galactose binding is believed to confer to the virus the ability to cross the blood-brain barrier (BBB) and infect the cells of the CNS including primary neurons.

According to the invention, a suitable AAV capsid is one of AAV-PHP, AAV9, AAV-BR1, AAV-Retro capsid, scAAV, AAV1, AAV2, AAV4, AAV5, AAV6, AAV7, AAV8, AAV9, AAV10, AAV11, AAV12, AAV13, AAV-PHP.B, AAV-PHP.eB, AAV-BR1, AAV-Retro, scAAV, AAVrh.8, AAVrh. 10, AAV.miRNA AAV-DJ, AAV-LK03, AAV2.7m8.

In an embodiment of the invention said AAV-PHP capsid is AAV-PHP.B, AAV-PHP.eB, AAV-PHP.S or AAV-PHP.A. AAV-PHP capsid proteins (and related capsids) are well-known genetically engineered AAV capsid protein created based on AAV serotype 9 capsid.

According to the invention, the nucleotide sequence packaged into the viral particle codes for a therapeutic protein, isoform or homolog thereof as defined above and in the claims, for the treatment of polyQ diseases as already defined in Table 1, a non limiting example of said therapeutic protein, isoform or homolog thereof is provided in Tables 2-4.

According to the present invention said therapeutic protein, isoform or homolog thereof is capable, when expressed by gene therapy, of reducing the toxicity of mutated pathogenic protein expressed by one or more of ATN1, HTT, AR, ATXN1, ATXN2, ATXN3, CACNA1A, ATXN7, PPP2R2B, TBP genes, wherein said pathogenic protein comprise a pathological number of polyQ residues.

According to the invention said therapeutic protein, isoform or homolog thereof is one of tables 2, 3 or 4.

An object of the invention is an expression vector, or delivery system or a vector suitable for gene therapy or an adeno-associated viral vector AAV or a Lentiviral vector, a nanoparticle, a LNP, a nucleotide sequence, preferably a gene therapy expression vector, even more preferably a gene therapy expression adeno-associated viral vector, AAV, or a viral particle as previously defined in the description and as defined in the claims, for use in the treatment by gene therapy of a polyQ disease.

According to the invention, hence, said polyQ disease is selected from Dentatorubropallidoluysian atrophy, Huntington's disease, Spinal and bulbar muscular atrophy, Spinocerebellar ataxia Type 1, Spinocerebellar ataxia Type 2, Spinocerebellar ataxia Type 3, Spinocerebellar ataxia Type 6, Spinocerebellar ataxia Type 7, Spinocerebellar ataxia Type 12 or Spinocerebellar ataxia Type 17.

In a preferred embodiment, said disease is Huntington's disease and said therapeutic protein, isoform or homolog thereof reduces the toxicity of the pathogenic protein produced by the mutated HHT gene as depicted in Table 1.

The invention also encompasses a method for the treatment of said polyQ diseases, preferably of Huntington's disease comprising the administration, to a subject in need thereof, of a therapeutically effective amount of the vector or of the viral particle according to any of the embodiments herein described and to the claims.

All definitions and examples of gene therapy methods/uses provided for the gene therapy vector of the invention apply, mutatis mutandis, to the viral particle of the invention.

The invention also relates to a nucleotide sequence, such as a cDNA or an mRNA molecule, optionally complexed with one or more carrier molecules, coding for a therapeutic protein, an isoform or a homologs thereof as herein defined, capable of reducing the toxicity of a mutated protein causing a polyQ disease (Table 1), preferably a therapeutic protein, isoform or homologs thereof as disclosed in Tables 2-4.

Preferably, said mRNA comprises a 3′ and a 5′ UTR element flanking the coding sequence, a 5′ Cap and a polyA tail. 5′ and 3′ mRNA untranslated regions (UTR) are well known in molecular genetics. 5′ UTR is a sequence containing a Kozak consensus sequence that is recognised by the ribosome and allows the ribosome to bind to the mRNA molecule and to initiate its translation. 3′ UTR region is found after the stop codon and has a role in translation termination and post-transcriptional modifications. UTRs are well known to the skilled person as well as is their role for enhanced protein production in non-viral gene therapies. The skilled person can readily select among UTRs commonly used in non-viral gene therapy a suitable 5′ and 3′ sequence for the mRNA construct of the present invention.

Additionally, the mRNA construct may also comprise a 5′ cap and a polyA tail. Both stabilise the mRNA molecule and increase protein translation.

Various versions of 5′ caps known in the art can be added during or after the transcription reaction using a vaccinia virus capping enzyme or by incorporating synthetic cap or anti-reverse cap analogues.

In addition, the mRNA molecule of the invention can comprise optimised codons, selected in order to match with the more abundant tRNAs of the organism to which said mRNA will be administered. In a preferred embodiment the codons can be optimised for humans.

Still, according to the present invention, the mRNA molecule, in any of the embodiments herein disclosed, can comprise one or more modified nucleosides in order to enhance the stability, the safety and/or the translation of the same. Suitable examples of modified nucleosides include but are not limited to pseudouridine and 1-methylpseudouridine.

Still in another embodiment, that applies to all the embodiments related to the mRNA molecule of the invention, said molecule can be naked or can be complexed with one or more carrier molecules according to the common knowledge in the art in the form, by way of example, of a in a cationic nanoemulsion, nanoparticle, liposome, cationic polymer liposome, polysaccharide particle cationic lipid nanoparticle, cationic lipid cholesterol nanoparticle or cationic lipid cholesterol PEG nanoparticle.

Commonly used delivery methods and carrier molecules for therapeutic mRNA molecules include: naked mRNA (part a); naked mRNA with in vivo electroporation; protamine (cationic peptide)-complexed mRNA; mRNA associated with a positively charged oil-in-water cationic nanoemulsion; mRNA associated with a chemically modified dendrimer and complexed with polyethylene glycol (PEG)-lipid; protamine-complexed mRNA in a PEG-lipid nanoparticle; mRNA associated with a cationic polymer such as polyethylenimine (PEI); mRNA complexed with a cationic polymer such as PEI and a lipid component; mRNA complexed with a polysaccharide (for example, chitosan) particle or gel; mRNA in a cationic lipid nanoparticle (for example, 1,2-dioleoyloxy-3-trimethylammoniumpropane (DOTAP) or dioleoylphosphatidylethanolamine (DOPE) lipids); mRNA complexed with cationic lipids and cholesterol (part k); and mRNA complexed with cationic lipids, cholesterol and PEG-lipid.

The cationic peptide protamine has been shown to protect mRNA from degradation by serum RNases.

According to a preferred embodiment of the invention, said mRNA molecule codes for one or more therapeutic proteins selected from metal regulatory transcription factor 1, lysine-specific demethylase 5B, lysine-specific demethylase 2B, F-box only protein 34, essential MCU regulator, mitochondrial, Ephrin type-A receptor 4, Transducin-like enhancer protein 4, Arfaptin-1, AT-rich interactive domain-containing protein 5B (Table 2) or an isoform or a homolog thereof as previously defined (e.g. Tables 3 and 4).

Still, according to an embodiment of the invention, said polyQ disease is Dentatorubropallidoluysian atrophy, Huntington's disease, Spinal and bulbar muscular atrophy, Spinocerebellar ataxia Type 1, Spinocerebellar ataxia Type 2, Spinocerebellar ataxia Type 3, Spinocerebellar ataxia Type 6, Spinocerebellar ataxia Type 7, Spinocerebellar ataxia Type 12 or Spinocerebellar ataxia Type 17 (see Table 1).

In a preferred embodiment of the invention the mRNA molecule in any of the embodiments provided above is advantageously for use in a gene therapy treatment of a polyQ disease as defined in Table 1. In a preferred embodiment, the poluQ disease is Hungtinton's disease.

The invention also relates to a method of treatment of said diseases comprising administering to a subject in need thereof the mRNA molecule as defined above in a therapeutically effective amount.

Another object of the invention is a pharmaceutical composition comprising or consisting of the therapeutic protein, isoform or homolog thereof as defined above or in the claims or an expression vector or delivery system or vector suitable for gene therapy, or adeno-associated viral vector AAV or Lentiviral vector, or nanoparticle, or LNP or nucleotide sequence as defined above or in the claims, or a viral particle as defined above or in the claims or a cDNA or mRNA as defined above or in the claims and a pharmaceutically acceptable carrier. When mRNA is used, the molecule can be formulated with a transfection reagent. Suitable transfection reagents are commercially available. The composition is a composition suitable for systemic (including intravenous) injection, central nervous system delivery or aerosol/nasal delivery. By way of example the injection can be intravenous injection, intraparenchymal administration in particular areas of the brain such as intracerebroventricular, cisternal, lumbar or intrathecal administration, or intra-arterial injection (carotid artery injection may be used for the delivery), or by direct administration into the cerebrospinal fluid.

Suitable pharmaceutical carriers for the administrations indicated above are well-known to the skilled person, by way of example saline solution can be used.

When intended for medical treatment every product herein described will be in sterilized form.

A further embodiment of the invention is hence the pharmaceutical composition f as defined in the description and in the claims and a pharmaceutically acceptable carrier, for use in the treatment of a polyQ disease.

According to the invention said polyQ disease is Dentatorubropallidoluysian atrophy, Huntington's disease, Spinal and bulbar muscular atrophy, Spinocerebellar ataxia Type 1, Spinocerebellar ataxia Type 2, Spinocerebellar ataxia Type 3, Spinocerebellar ataxia Type 6, Spinocerebellar ataxia Type 7, Spinocerebellar ataxia Type 12 or Spinocerebellar ataxia Type 17.

In a preferred embodiment, said polyQ disease is Huntington's disease.

A further object of the invention is therefore a medical treatment comprising administering therapeutically effective doses of the therapeutic protein, isoform or homolog thereof as defined above or in the claims or the expression vector or delivery system or vector suitable for gene therapy, or adeno-associated viral vector AAV or Lentiviral vector, or nanoparticle, or LNP or nucleotide sequence as defined above or in the claims, or the viral particle as defined above or in the claims or the cDNA or mRNA as defined above or in the claims or the pharmaceutical composition of the invention to a patient in need thereof.

In addition, the present invention relates also to the use of the therapeutic protein, isoform or homolog thereof as defined above or in the claims or the expression vector or delivery system or vector suitable for gene therapy, or adeno-associated viral vector AAV or Lentiviral vector, or nanoparticle, or LNP or nucleotide sequence as defined above or in the claims, or the viral particle as defined above or in the claims or the cDNA or mRNA as defined above or in the claims, for the manufacture of a medicament for the treatment of a polyQ disease of Table 1, in particular of Hungtinton's disease.

In said embodiment, a the therapeutic protein, isoform or homolog thereof as defined above or in the claims or the expression vector or delivery system or vector suitable for gene therapy, or adeno-associated viral vector AAV or Lentiviral vector, or nanoparticle, or LNP or nucleotide sequence as defined above or in the claims, or the viral particle as defined above or in the claims or the cDNA or mRNA as defined above or in the claims are formulated with one or more suitable pharmaceutical carriers and/or excipients, that is selected by the skilled person from the commonly used for the desired formulation (refer to pharmaceutical composition above) for the treatment of a polyQ disease of table 1.

The invention can be hence summarised in the following items:

1. A therapeutic protein, or an isoform or an homolog thereof, said homolog having a sequence homology, defined as at least 55% coverage and at least 49% identity with said therapeutic protein, wherein said therapeutic protein, isoform or homolog thereof is capable of reducing the toxicity of a mutated protein causing a polyQ disease, for use in the treatment of said disease.

2. The therapeutic protein or isoform or homolog thereof, for use according to claim 1 wherein said mutated protein is expressed by one or more of ATN1, HTT, AR, ATXN1, ATXN2, ATXN3, CACNA1A, ATXN7, PPP2R2B, TBP genes, said mutated protein comprising a pathological number of polyQ residues.

3. The therapeutic protein for use according to items 1 or 2 wherein said therapeutic protein is selected from metal regulatory transcription factor 1, lysine-specific demethylase 5B, lysine-specific demethylase 2B, F-box only protein 34, essential MCU regulator, mitochondrial, Ephrin type-A receptor 4, Transducin-like enhancer protein 4, Arfaptin-1, AT-rich interactive domain-containing protein 5B.

4. The therapeutic protein isoform for use according to items 1 or 2 wherein said isoform is selected from Table 3 below

| Therapeutic protein | |
|---|---|
| from table 2 | Isoforms |
| MTF1 | NM_005955.3; XM_011541494.1; XM_011541491.2; XM_011541493.3 |
| KDM5B | NM_001314042.1; NM_001347591.1; NM_006618.5 |
| KDM2B | NM_001005366.2; NM_032590.5; XM_011538867.3; XM_011538868.3; |
| | XM_005253955.4; XM_005253956.4; XM_005253961.5; XM_011538875.3; |
| | XM_011538869.2 |
| FBX034 | NM_017943.4; NM_152231.2; XM_006720185.3; XM_017021391.1; |
| | XM_017021392.1; XM_017021393.2; XM_005267791.4 |
| SMDT1 | NM_033318.5; NR_146715.2; XM_011530509.3; NR_146717.1 |
| EPHA4 | NM_001304536.2; NM_001304537.2; NM_001363748.2; NM_004438.5 |
| TLE4 | NM_001282748.2; NM_001282749.2; NM_001282753.2; NM_001282760.2; |
| | NM_001351541.2; NM_001351542.2; NM_001351543.2; NM_001351546.2; |
| | NM_001351547.2; NM_001351550.2; NM_001351552.2; NM_001351556.2; |
| | NM_001351558.2; NM_001351560.2; NM_001351562.2; NM_001351563.2; |
| | NM_001351564.2; NM_007005.6; NR_104239.2; XM_011518952.1; |
| | XM_006717264.1; XM_011518958.1; XM_006717268.1; XM_011518967.1; |
| | XM_011518969.1; XM_011518956.1; XM_011518957.1; XM_011518959.1; |
| | XM_011518965.1; XM_017015081.1; XM_011518960.1; XM_011518966.1; |
| | XM_017015083.1; XM_011518963.1; XM_011518964.1; XM_011518955.1; |
| | XM_011518961.1; XM_011518962.1; XM_011518968.1; XM_011518954.1; |
| | XM_017015068.1; XM_017015075.1; XM_011518970.3; XM_017015070.1; |
| | XM_017015067.1; XM_011518953.2; XM_017015069.2; XM_011518972.3; |
| | XM_024447659.1 |
| ARFIP1 | NM_001025593.3; NM_001025595.3; NM_001287431.2; NM_001287432.2; |
| | NM_001287433.2; NM_014447.4 |
| ARID5B | NM_001244638.2; NM_032199.3 |

5. The therapeutic protein homolog for use according to items 1 or 2 wherein said homolog is selected from Table 4 below

| Homologs | REFERENCE NCBI |
|---|---|
| KDM5A | 5927 |
| KDM5C isoform 3 | NP_001269551.1 |
| FBXL11 | 22992 |
| EPHA5 isoform d and b | NP_001268695.1 |
| | NP_872272.2 |
| EPHA5 isoform a and c | NP_004430.4 |
| | NP_001268694.1 |
| EPHA7 isoform 2 precursor | NP_001275558.1 |
| EPHA7 isoform 5 precursor | NP_001363395.1 |
| EPHA7 isoform 1 precursor | NP_004431.1 |
| EPHA7 isoform 4 precursor | NP_001363394.1 |
| EPHA5 isoform f | NP_001305690.1 |
| EPHA5 isoform e | NP_001268696.1 |
| EPHA3 isoform a precursor | NP_005224.2 |
| EPHA3 isoform X1 | XP_005264772.1 |
| TLE3 isoform e | NP_001269908.1 |
| TLE3 isoform X5 | XP_005254680.1 |
| TLE3 isoform d | NP_001269909.1 |
| TLE3 isoform b | NP_001098662.1 |
| TLE3 isoform X7 | XP_011520282.1 |
| TLE3 isoform X4 | XP_011520280.1 |
| TLE3 isoform X3 | XP_005254679.1 |
| TLE3 isoform a | NP_005069.2 |
| TLE3 isoform X2 | XP_011520279.1 |
| TLE3 isoform X1 | XP_011520278.1 |
| TLE3 isoform f | NP_001269910.1 |
| TLE3 isoform X15 | XP_005254690.1 |
| TLE3 isoform X10 | XP_005254685.1 |
| TLE3 isoform c | NP_065959.1 |
| TLE3 isoform X8 | XP_005254682.1 |
| TLE1 isoform X12 | XP_006717323.1 |
| ARFIP2 isoform 9 | NP_001363487.1 |
| ARFIP2 isoform 5 | NP_001357335.1 |
| ARFIP2 isoform 10 | NP_001363489.1 |
| ARFIP2 isoform 1 | NP_001229783.1 |
| ARFIP2 isoform 8 | NP_001357341.1 |
| ARFIP2 isoform 7 | NP_001357338.1 |

6. The therapeutic protein, isoform or homolog thereof, for use according to anyone of items 1 to 5 wherein said polyQ disease is Dentatorubropallidoluysian atrophy, Huntington's disease, Spinal and bulbar muscular atrophy, Spinocerebellar ataxia Type 1, Spinocerebellar ataxia Type 2, Spinocerebellar ataxia Type 3, Spinocerebellar ataxia Type 6, Spinocerebellar ataxia Type 7, Spinocerebellar ataxia Type 12 or Spinocerebellar ataxia Type 17.

7. The therapeutic protein, isoform or homolog thereof, for use according to item 6 wherein said disease is Huntington's disease.

8. The therapeutic protein, isoform or homolog thereof, for use according to anyone of items 1 to 5 wherein said therapeutic protein, isoform or homolog thereof, is expressed by: an expression vector or delivery system, or a vector suitable for gene therapy or an adeno-associated viral vector AAV or a Lentiviral vector, or a nanoparticle, or a LNP, or a nucleotide sequence . . .

9. An expression vector or delivery system or a vector suitable for gene therapy, or an adeno-associated viral vector AAV or a Lentiviral vector, or a nanoparticle, or an LNPcomprising a nucleotide sequence coding for a therapeutic protein, isoform or homolog thereof, said isoform or homolog having a sequence homology defined as at least 55% coverage and at least 49% identity with said therapeutic protein, wherein said therapeutic protein, isoform or homolog thereof is capable of reducing the toxicity of a mutated protein causing a polyQ disease,

10. The expression vector or delivery system or vector suitable for gene therapy, or adeno-associated viral vector AAV or Lentiviral vector, or nanoparticle, or LNP according to item 9 wherein said mutated protein expressed by one or more of ATN1, HTT, AR, ATXN1, ATXN2, ATXN3, CACNA1A, ATXN7, PPP2R2B, TBP genes, wherein said protein comprises a pathological number of polyQ residues.

11. The expression vector or delivery system or vector suitable for gene therapy, or adeno-associated viral vector AAV or Lentiviral vector, or nanoparticle, or LNP according to items 9 or 10 wherein said nucleotide sequence codes for a therapeutic protein, isoform or homolog thereof as defined in items 3 to 5.

12. The vector according to anyone of items from 9 to 11 wherein said vector is a viral vector AAV having an optimal capsid serotype for CNS gene therapy.

13. The viral vector AAV according to item 12 wherein said capsid serotype is one of: AAV1, AAV2, AAV4, AAV5, AAV6, AAV7, AAV8, AAV9, AAV10, AAV11, AAV12, AAV13, AAV-PHP.B, AAV-PHP.eB, AAV-BR1, AAV-Retro, scAAV, AAVrh.8, AAVrh. 10, AAV.miRNA AAV-DJ, AAV-LK03, AAV2.7m8.

14. An expression vector or delivery system or vector suitable for gene therapy, or adeno-associated viral vector AAV or Lentiviral vector, or nanoparticle, or LNP or nucleotide sequence as defined in anyone of items 9 to 13, for use in the treatment of a polyQ disease.

15. The expression vector or delivery system or vector suitable for gene therapy, or adeno-associated viral vector AAV or Lentiviral vector, or nanoparticle, or LNP or nucleotide sequence for use according to item 14 wherein said polyQ disease is Dentatorubropallidoluysian atrophy, Huntington's disease, Spinal and bulbar muscular atrophy, Spinocerebellar ataxia Type 1, Spinocerebellar ataxia Type 2, Spinocerebellar ataxia Type 3, Spinocerebellar ataxia Type 6, Spinocerebellar ataxia Type 7, Spinocerebellar ataxia Type 12 or Spinocerebellar ataxia Type 17.

16. The expression vector or delivery system or vector suitable for gene therapy, or adeno-associated viral vector AAV or Lentiviral vector, or nanoparticle, or LNP or nucleotide sequence for use according to item 15 wherein said polyQ disease is Huntington's disease.

17. A viral particle comprising a viral capsid and a nucleotide sequence coding for the therapeutic protein or isoform or homolog thereof as defined in any one of items 1 to 5.

18. The viral particle according to item 17 wherein said capsid is an AAV capsid having an optimal serotype for CNS gene therapy.

19. The viral particle according to item 18 wherein said AAV capsid is one of AAV1, AAV2, AAV4, AAV5, AAV6, AAV7, AAV8, AAV9, AAV10, AAV11, AAV12, AAV13, AAV-PHP.B, AAV-PHP.eB, AAV-BR1, AAV-Retro, scAAV, AAVrh.8, AAVrh. 10, AAV.miRNA AAV-DJ, AAV-LK03, AAV2.7m8.

20. The viral particle according to item 19 wherein said AAV-PHP capsid is AAV-PHP.B, AAV-PHP.eB, AAV-PHP.S or AAV-PHP.A.

21. The viral particle according to any one of items 17 to 20 for use in a gene therapy treatment of a polyQ disease.

22. The viral particle for use according to item 21 wherein said said polyQ disease is Dentatorubropallidoluysian atrophy, Huntington's disease, Spinal and bulbar muscular atrophy, Spinocerebellar ataxia Type 1, Spinocerebellar ataxia Type 2, Spinocerebellar ataxia Type 3, Spinocerebellar ataxia Type 6, Spinocerebellar ataxia Type 7, Spinocerebellar ataxia Type 12 or Spinocerebellar ataxia Type 17.

23. The viral particle for use according to item 22 wherein said polyQ disease is Huntington's disease.

24. An mRNA coding for a therapeutic protein, isoform or homolog thereof, said isoform or homolog having a sequence homology defined as at least 55% coverage and at least 49% identity with said therapeutic protein, wherein said therapeutic protein, isoform or homolog thereof is capable of reducing the toxicity of a mutated protein causing a polyQ disease.

25. The mRNA according to item 24 wherein said mutated protein is expressed by one or more of ATN1, HTT, AR, ATXN1, ATXN2, ATXN3, CACNA1A, ATXN7, PPP2R2B, TBP genes, wherein said protein comprises a pathological number of polyQ residues.

26. The mRNA according to item 24 or 25 coding for a therapeutic protein, isoform or homologs thereof as defined in items 3 to 5.

27. The mRNA according to anyone of items 24 to 26, wherein said mRNA comprises a 3′ and a 5′ UTR element flanking the coding sequence, a 5′ Cap and a polyA tail.

28. The mRNA according to anyone of items 24 to 27, wherein said mRNA comprises one or more modified nucleosides.

29. The mRNA according to anyone of items 24 to 28, wherein said mRNA is complexed with one or more carrier molecules.

30. The mRNA according to anyone of items 24 to 29, wherein said mRNA is complexed in a cationic nanoemulsion, in a nanoparticle, in a liposome, in a cationic polymer liposome, in a polysaccharide particle, in a cationic lipid nanoparticle, in a cationic lipid cholesterol nanoparticle, in a cationic lipid cholesterol PEG nanoparticle.

31. An mRNA according to anyone of items 24 to 30, for use in the treatment of a polyQ disease.

32. The mRNA for use according to item 31 wherein said polyQ disease is Dentatorubropallidoluysian atrophy, Huntington's disease, Spinal and bulbar muscular atrophy, Spinocerebellar ataxia Type 1, Spinocerebellar ataxia Type 2, Spinocerebellar ataxia Type 3, Spinocerebellar ataxia Type 6, Spinocerebellar ataxia Type 7, Spinocerebellar ataxia Type 12 or Spinocerebellar ataxia Type 17.

33. The mRNA for use according to item 32 wherein said polyQ disease is Huntington's disease.

34. A pharmaceutical composition comprising the therapeutic protein, isoform or homolog thereof as defined in items 1 to 5, or an expression vector or delivery system or vector suitable for gene therapy, or adeno-associated viral vector AAV or Lentiviral vector, or nanoparticle, or LNP or nucleotide sequence according to anyone of items 8-13, or a viral particle according to any one of items 17 to 20 or a mRNA according to anyone of items 24 to 30 and a pharmaceutically acceptable carrier.

35. A pharmaceutical composition according to item 34 for use in the treatment of a polyQ disease.

36. The pharmaceutical composition for use according to item 35 wherein said polyQ disease is Dentatorubropallidoluysian atrophy, Huntington's disease, Spinal and bulbar muscular atrophy, Spinocerebellar ataxia Type 1, Spinocerebellar ataxia Type 2, Spinocerebellar ataxia Type 3, Spinocerebellar ataxia Type 6, Spinocerebellar ataxia Type 7, Spinocerebellar ataxia Type 12 or Spinocerebellar ataxia Type 17.

37. The pharmaceutical composition for use according to item 36 wherein said polyQ disease is Huntington's disease.

39. The pharmaceutical composition according to item 35 for administration by systemic injection, central nervous system delivery or aerosol/nasal delivery.

40. The pharmaceutical composition according to item 39 for intravenous injection administration, intraparenchymal administration in particular areas of the brain such as intracerebroventricular, cisternal, lumbar or intrathecal administration, or intra-arterial injection administration, or for direct administration into the cerebrospinal fluid.

TABLE 5

| Organism | Gene | Forward primer sequence | Reverse primer sequence |
|---|---|---|---|
| Mouse | Arid1b | CTGGACCTGTTCCGACTGTA | CAACGTTCAGGTTGGTTGG |
| | | SEQ ID NO 1 | SEO ID NO 2 |
| Mouse | Fbxo34 | CCTCCCAGTCTTGAGTCAGC | CATAGGCGTTCTCAGGGTGT |
| | | SEQ ID NO 3 | SEQ ID NO 4 |
| Mouse | Gapdh | ATCCTGCACCACCAACTGCT | GGGCCATCCACAGTCTTCTG |
| | | SEQ ID NO 5 | SEQ ID NO 6 |
| Human/Mouse | Htt | TGATGGATTCTAATCTTCCAAGG | GTGAGCCAGCTCAGCAAAC |
| | | SEQ ID NO 7 | SEQ ID NO 8 |
| Mouse | Kdm2b | CTGCTCTCGAAGCTCCAACT | TTTCCACAGCGTTTGAAGAA |
| | | SEQ ID NO 9 | SEO ID NO 10 |
| Mouse | Kdm5b | ACCTGACCTCCGACACAAAG | TGGCTTCTGTTGCCTCTTCT |
| | | SEQ ID NO 11 | SEQ ID NO 12 |
| Mouse | Kdm5b- | ATCTGTGTGCGCTGTACTGG | GCGATGCAATTTCCTCATTT |
| | UTR | SEQ ID NO 13 | SEO ID NO 14 |
| Mouse | MT1 | CTGCTGCTCCTGCTGTCC | CAGCGCTGTTCGTCACAT |
| | | SEQ ID NO 15 | SEQ ID NO 16 |
| Mouse | MT2 | GCAAATGCAAACAATGCAAA | CACTTGTCGGAAGCCTCTTT |
| | | SEQ ID NO 17 | SEO ID NO 18 |
| Mouse | Mtf1 | GAAGCAATGTCCCAGGGTTA | CTCCTCGGTGAGTCTTCTGG |
| | | SEQ ID NO 19 | SEQ ID NO 20 |
| | AAV_Mtf1 | AGCAGATTGGCCTCAGTGTT | TCCATCACTAGGGGTTCCTG |
| | | SEQ ID NO 21 | SEQ ID NO 22 |
| Mouse | β-Actin | GTTGACATCCGTAAAGACC | TGGAAGGTGGACAGTGAG |
| | | SEQ ID NO 23 | SEQ ID NO 24 |
| Mouse | Synj2 | CAGGAGGCAGAAGCAGCTAT | TGGCTTAGAAGACCCTTGGTT |
| | | SEQ ID NO 25 | SEQ ID NO 26 |
| | eGFP | TATATCATGGCCGACAAGCA | ACTGGGTGCTCAGGTAGTGG |
| | | SEQ ID NO 27 | SEQ ID NO 28 |
| Zebrafish | B2m | GCCTTCACCCCAGAGAAAGG | CCGGTTTGGATTTACATGTTG |
| | | SEQ ID NO 29 | SEQ ID NO 30 |
| Zebrafish | Eef1a | GACAAGAGAACCATCGAG | CTGAAACTCACCGACAC |
| | | SEQ ID NO 31 | SEQ ID NO 32 |
| Zebrafish | Gapdh | GTGGAGTCTACTGGTGTCTTC | GTGCAGGAGGCATTGCTTACA |
| | | SEO ID NO 33 | SEO ID NO 34 |
| Zebrafish | Tuba1b | CCCACTGTCATTGATGAGG | CGAACCCTATCTAAAACAAGGT |
| | | SEQ ID NO 35 | CA SEQ ID NO 36 |

The following examples demonstrate the therapeutic effectiveness in cell and/or animal models of the therapeutic proteins identified by the inventors and listed in table 2. These examples are provided to show completely one way to carry the invention and the observed results in vivo.

All experiments on animals were performed in accordance with the Italian and European Community law for the use of experimental animals and were approved by Bicocca University Bioethical Committee.

Embryonic Stem (ES) cells herein named “Rex1GFP-d2” and “E14TG2a” were obtained from mouse blastocyst embryos and were gifts from the Austin Smith laboratory (University of Exeter-UK).

### Examples

### Establishment and Characterization of Mutant HTT-Expressing ES Cells

Embryonic Stem (ES) cells herein named “Rex1GFP-d2” and “E14TG2a” were obtained from mouse blastocyst embryos and were gifts from the Austin Smith laboratory (University of Exeter-UK).

ES cells stably expressing an N-terminal fragment of either mutant (containing 128 CAG repeats) or wild-type (15 CAG repeats) HTT, named Q15 and Q128 cells, respectively (FIG. 1a and FIG. 7a) were generated. Expression of mutant HTT did not alter the expression of pluripotency markers (FIG. 7a, right). Translation of mutant and wild type form of Q128 and Q15 HTT protein in cells was confirmed by Western Blot (FIG. 1b). The cell toxicity induced by the expression of Q128 HTT was verified. We measured the number of cells obtained over 4 days of culture and observed a pronounced reduction of Q128 cells relative to Q15 cells (FIG. 1c). Conversely, Q15 cells expanded robustly. Similar results were obtained with an independent parental ES cell line (FIG. 7b-d). Moreover, Q128 cells displayed increased cell death (FIG. 1d and FIG. 7e) and increased production of Reactive Oxygen Species (ROS, FIG. 1e and FIG. 7f) as previously reported in the art. Collectively, these data demonstrated that expression of Q128 HTT, but not Q15 HTT, impaired ES cells viability.

To further validate the Q128 HTT expressing ES cell model, transcriptome analysis was carried out and differentially expressed genes (DEGs) in Q128 versus Q15 cells were identified (FIG. 1f). Gene list enrichment analysis (FIG. 1g) identified misregulation of genes associated with processes implicated in HD pathogenesis, such as metabolism, transcriptional regulation, post-translational modification such as ubiquitylation, methylation, acetylation, and metal homeostasis. Moreover, among the DEGs we found several genes previously associated with Huntington's disease as biomarkers or because of their altered expression in HD models, such as Dpf1, Med29, Stub1, Polr3b, Psmc1, Setdb1, Ube2cbp, Pqbp1 and Prnp (FIG. 1f). Q128 cells, hence, recapitulate molecular features associated with HD.

### A Gain-of-Function Screen for Suppressors of Mutant HTT Toxicity

With the aim of performing a genetic screening, experimental conditions that would exacerbate the toxic effects of mutant HTT, in order to facilitate the isolation of fully resistant mutants was searched. The inventors reasoned that by treating Q128 cells with different cell stressors we could induce their death, allowing the identification of genetic mutants resistant to the toxic effects of mutant HTT (FIG. 2a). Small molecules and inhibitors that were previously shown to worsen the effect of mutant HTT acting on biological processes implicated in HD, such as autophagy, the proteasomal degradation system or mitochondrial metabolism were searched and a panel of inhibitors was selected and titrated in order to find doses that were not lethal in parental ES cells (FIG. 8a). Next, Q128 cells were treated with the inhibitors and it was observed that MG132 and Tamoxifen further reduced viability of Q128 cells (FIG. 2b), therefore they were chosen as stressors selectively inducing cell death in Q128 cells to be used for the genetic screening (FIG. 2a). Two independent stressors, affecting unrelated biological processes, were used in order to better identify candidate genes acting specifically on mutant HTT, rather than on the stressors themselves.

For the screening, a gain-of-function approach was selected. This has the advantage that candidates are selected among all genes present in the intact genome of ES cells, not only among genes expressed in ES cells or expressed in response to mutant HTT, as for loss-of-function approaches, thus widening our potential for discovery. To generate the genetic mutants, PB vectors that are based on transposons, DNA elements that stably integrate into the genome, were used. Electroporation of a PB vector in the presence of the transposase leads to random integration into TTAA sites that are abundant in the genome. The PB vector used (pGG134, FIG. 2a) was optimized for gain-of-function screens in ES cells and consists of the murine stem cell virus (MSCV) enhancer/promoter followed by a splice donor site, which allows the over-activation of endogenous genes flanking the site of integration. We electroporated Q128 cells with the pGG134 vector or a PB vector encoding for GFP (PB_GFP), which served as a control. After treatment with exogenous stressors for 5 days, the surviving colonies were counted. Parental ES cells expressing GFP, serving as controls, robustly proliferated in the presence of stressors, while very few Q128 cells expressing GFP survived MG132 or Tamoxifen treatment (FIG. 2c). In contrast, Q128 cells electroporated with pGG134 formed significantly more resistant colonies in presence of both stressors (FIG. 2c), indicating the successful generation of mutants resistant to Q128 HTT. A total of 44 mutant clonal lines emerged from 4 mutagenesis experiments were individually picked and expanded and named MG # or T #, according to their derivation in presence of the stressors MG132 or Tamoxifen, respectively. In order to identify which gene was activated in individual clones, Splinkerette-PCR (Sp-PCR), which allowed amplification of genomic regions flanking the site of integration was carried out. For 35 clones 1 or 2 major bands corresponding to the genomic regions at the 5′ and 3′ ends of the PB vector (FIG. 2d, see MG15) were obtained. Sp-PCR bands were then excised and sequenced. Sequence alignment to the reference genome allowed the identification of the precise site of integration and orientation in each mutant cell line: in the example shown in FIG. 2d, the pGG134 was found inserted upstream of the Kdm5b gene and in the correct orientation allowing its activation (FIG. 2d, bottom panel). 23 genes flanking pGG134 (complete list of integration sites in FIG. 8b) representing genes potentially conferring resistance to mutant HTT, or “suppressors” were identified.

The integration of the PB vector in the proximity of a given gene should lead to its overexpression.

It was verified by qPCR that endogenous Kdm5b transcripts appeared upregulated in the MG15 clone compared to Q128 cells electroporated with PB_GFP or to parental ES cells (FIG. 2e). 4 additional mutant clones, whose integration sites were unequivocally identified and corresponded to Fbxo34 gene (MG21 clone), Mtf1 (MG18), Synj2 (MG9) and Arid1b (T4) were characterized. In each clonal line, the upregulation of the cognate genes relative to controls (FIG. 2e) was verified. To validate the ability of the screening procedure to select for mutants resistant to toxicity induced by Q128, the five selected clones were exposed to both exogenous stressors individually. All clones survived comparably to parental ES cells, while Q128 cells electroporated with PB_GFP vector showed highly reduced survival (FIG. 2f and FIG. 8c). Clonal lines were resistant to both stressors suggesting that the mutagenesis procedure led to the activation of genes conferring resistance to mutant HTT, rather than resistance to MG132 or Tamoxifen themselves. As the clones might have acquired resistance to mutant HTT by simply silencing the Q128 HTT transgene, both HTT mRNA (FIG. 8d) and protein (FIG. 8e), which were robustly detected in all clones were monitored. The screening procedure, hence, led to the identification of 5 genes as bona fide suppressors of toxicity induced by mutant HTT.

### FIG. 8

### Secondary Validation of Mutant HTT Suppressors

Next, Q128 cells with a vector containing cDNA of candidate genes under the control of a constitutive promoter were transfected, with the aim of confirming that the identified candidates (therapeutic proteins) were able to confer resistance to mutant HTT (FIG. 3a). For such validation experiments, Mtf1, Kdm5b, and Fbxo34, which were identified in mutant clones were selected and Kdm2b, which was identified multiple times in the genome-wide screening (FIG. 3b). Firstly, it was checked whether the expression of Mtf1, Kdm2b, Kdm5b and Fbxo34 was indeed increased in cells expressing each candidate, named Q128_Mtf1, Q128_Kdm2b, Q128_Kdm5b and Q128_Fbxo cells, respectively. High levels of candidate genes expression in all cell lines generated relative to relevant controls (FIG. 3b) were observed, while HTT Q128 levels were unaltered (FIG. 3c and FIG. 10a). Finally, it was checked whether the proliferation of Q128 expressing cells was affected by the co-expression of the selected therapeutic proteins. Expression of Fbxo34 or Kdm5b had a very mild effect, while increased levels of Mtf1 and Kdm2b led to increased proliferation of Q128 cells (FIG. 3d). The cells were then exposed to stressors MG132 or Tamoxifen and, among all candidates, Mtf1 stood out for its capacity to confer resistance to mutant HTT in presence of both stressors (FIG. 3e). It was concluded that expression of Mtf1 consistently conferred resistance to mutant HTT, confirming the phenotype observed in the clone in which this gene was found upregulated, therefore it was chosen for further molecular characterization and in vivo studies.

### Mtf1 Regulates HD-Related Genes

MTF1 is a transcription factor that acts as a sensor for various stress conditions in the cell. Upon accumulation of metals (such as Cadmium, Zinc or Iron) but also hypoxia or oxidative stress, MTF1 translocates into the nucleus and activates the transcription of a set of genes, including transporters of metals and endogenous metal chelators called metallothioneins (MTs).

Given that MTF1 is a transcription factor, RNAseq to identify the transcriptional program controlled by Mtf1 conferring protection against mutant HTT was performed. It was first asked whether the DEGs regulated by mutant HTT (FIG. 1f) were affected by Mtf1 and observed that 32.78% (181 out of 552) were significantly rescued (FIG. 4a). Among the 258 genes upregulated by Q128, 66 were rescued, interestingly including the HD related genes Dpf1 and Ube2cbp (FIG. 4b). Similarly, 115 out of 294 genes downregulated by Q128 were rescued by Mtf1, including the HD related gene Prnp.

Next, the global transcriptional response elicited by Mtf1 in Q128 cells was analysed and it was found that Mt1 and Mt2, together with Mtf1 itself, were the genes most significantly upregulated (FIG. 4c). Gene list enrichment analysis revealed regulations of genes associated with metal homeostasis, such as the zinc transporters Slc30a1 and Slc30a2 and regulation of ROS (FIG. 4d). Importantly, Mtf1 did not induce any oncogenic gene signature and known regulators of cell proliferation or apoptosis were not affected. Indeed, no significant effects on cell proliferation rates were observed upon Mtf1 overexpression on parental ES cells or Q15 cells (FIG. 11a and FIG. 4e). It was concluded that Mtf1 controls the expression of HD-related genes and genes involved in metal homeostasis and ROS regulation in Q128 cells. Given that Q128 cells displayed increased cell death and ROS production (FIG. 1d and FIG. 1e), it was assessed whether Mtf1 overexpression could prevent those processes. Reduced cell death (FIG. 4f) and lower ROS production (FIG. 4g) in Q128_Mtf1 compared to Q128 cells was detected.

It was then assessed whether mutant HTT affects Mtf1 expression or activity. No changes in Mtf1 in Q128 cells (FIG. 3b) were observed. The expression levels of Mt1 and Mt2 as a proxy of Mtf1 activity (FIG. 4h and) were measured by qPCR and RNAseq and no significant differences in Q128 cells was found, indicating that endogenous Mtf1 is not activated in response to cytotoxic effects caused by mutant HTT. In contrast, Q128_Mtf1 cells displayed a highly robust induction of Mt1 and Mt2, associated with lower ROS production and reduced cell death (FIG. 4f-g). Overall, these results indicated that the presence of mutant HTT causes increased ROS production and cell death.

The endogenous Mtf1 pathway appears unable to counteract such effects, while expression of exogenous Mtf1 results in robust upregulation of the endogenous metal chelators Mt1/2, reduced ROS production and reduced cell death in Q128 cells.

### Mtf1 Counteracts Mutant HTT Effects in Zebrafish

Zebrafish is a powerful vertebrate model system widely used for human disease modelling, including HD. Therefore, it was decided to test whether Mtf1 would display protective effects in vivo using a Zebrafish HD model. We expressed in vivo Q16 and Q74 HTT fused in frame with eGFP through mRNA microinjection in fertilized eggs at the one-cell stage and characterised the phenotype of injected embryos (identified by GFP expression, FIG. 11a) at 24 hours post-fertilization (hdp) (FIG. 5a).

Injection of Q74eGFP at titrated doses (FIG. 11b) led to malformations as reduced body length and loss of cephalic structures. The injected larvae were divided into three main groups based on the severity of their phenotype (representative images of each group are shown in FIG. 5b, bright field panels) as Healthy embryos (H), embryos with Mild malformations (M), when embryos showed decreased head volume or cyclopia, a reduced body axis length and a curly tail, and embryos with Severe malformations(S), when embryos appeared as disorganized masses without distinguishable anterior and posterior regions. Finally, some embryos underwent rapid degeneration and widespread death and were classified as dead. Quantification of 8 independent experiments showed high percentages of mild, severe malformation and dead larvae upon Q74eGFP injections, while over 90% of Q16eGFP injected embryos were healthy (FIG. 5c), despite the same levels of the two mRNAs being detected in embryos (FIG. 5d). We concluded that only expression of mutant HTT in Zebrafish embryos impaired embryonic development of anterior structures.

Next, it was assessed whether embryonic degeneration involved cell death. Toward this aim, we performed Acridine Orange staining in 24 hpf microinjected embryos and detected regions of intense signal specifically in Q74eGFP embryos showing malformations (FIG. 5b, bottom panels). Increased cell death in Q74eGFP injected embryos was confirmed also by in situ terminal deoxynucleotidyl transferase (TdT)-mediated dUTP nick-end labelling (TUNEL) assay followed by confocal microscopy analysis and quantification. Control embryos injected with Q16eGFP mRNA showed TUNEL positivity (FIG. 5e) in body areas where apoptosis physiologically takes place at this stage of development, such as the optic vesicle, the diencephalon and the telencephalon. Conversely, Q74eGFP injected embryos displayed a widespread strong TUNEL-positive signal (FIG. 5e-f) especially in the severely misshapen anterior regions (64.2% of injected embryos, with a ˜5-fold increase in TUNEL-positive area). It was concluded that microinjection of mutant HTT in Zebrafish embryos leads to widespread cell apoptosis.

It was then assessed whether Mtf1 could suppress the detrimental effect of mutant HTT. Q74eGFP and Mtf1 mRNAs were co-injected and a reduction in the fraction of severely malformed (from 28% to 17%, p=0.002) and dead embryos was observed (from 16.5% to 8.5%, p=0.036, FIG. 5c) ultimately doubling the fraction of healthy embryos (from 20% to 46.5%, p=0.005). Crucially, Mtf1 expression led also to a marked decrease in Acridine orange signal intensity (FIG. 5b, bottom panels) and in TUNEL-positive areas (FIG. 5e-f), indicating decreased cell death.

### AAV-Vector Delivery of Mtf1 Alleviates Motor Deficit in R6/2 Mice

Observing protective effects of Mtf1 in a vertebrate model prompted us to test its function also in a more established HD model, such as the widely used R6/2 mice. The R6/2 mice display early HD-related phenotypes characterized by locomotor hyperactivity and learning impairment (roughly 3 weeks of age), followed by a progressive neurological degeneration leading to full manifestations around 8-15 weeks with severe motor coordination deficits. Such alterations are characterised by cell loss in the striatum and overall brain atrophy.

To express Mtf1 in the mouse brain, we used AAV-PHP.eB, a capsid that has been engineered to efficiently cross the blood-brain barrier upon intravenous injection. This viral vector diffuses over large neural areas including basal ganglia, resulting in transduction of >90% of neurons in the striatum upon a single administration in several mouse models. We first assessed whether the AAV-PHP.eB vectors we chose could efficiently cross the blood-brain barrier of R6/2 mice. To this aim, four-week old R6/2 mice were tail-vein injected with AAV-containing GFP and the viral expression was confirmed by immunoblotting. (FIG. 6b).

Next, R6/2 mice and WT littermates underwent to a single tail-vein injection of AAV-PHP.eB packaging either GFP (AAV-GFP), used as control, or Mtf1 (AAV-Mtf1), and motor performance was assessed weekly by Rotarod and Horizontal Ladder Task (HLT) as a functional readout of striatal neuronal loss 20,76 (FIG. 6a). R6/2 mice injected with AAV-GFP fell more rapidly than wild-type littermates in the Rotarod test from 7 weeks of age, as previously reported. R6/2 mice injected with AAV-Mtf1 maintained performances similar to wild-type littermates for the entire duration of the analysis (FIG. 6c). The HLT revealed an increased number of errors for R6/2 mice relative to wild-type mice at 7 weeks of age, as previously reported. Injection with AAV-Mtf1 rescued such effects (FIG. 6d). Finally, we detected reduced body weight of R6/2 mice compared to WT littermates when AAV-GFP was injected. Mtf1 had no significant effect on the body weight of R6/2 mice (FIG. 6e). In order to confirm expression of exogenous Mtf1, mice were sacrificed at end-point and striatal and cortical tissues were collected. PCR on total DNA robustly detected Mtf1 viral copies in both brain regions (FIG. 6f), thus confirming correct in vivo delivery of Mtf1. Altogether, these results indicated that injection of AAV-Mtf1 vector strongly and specifically ameliorated the motor defects observed in R6/2 mice.

### Methods

ES cell culture. ES cell lines (Rex1GFP-d2 and E14TG2a) were cultured in feeder free conditions (plastic coated with 0.2% gelatine [Sigma, cat. G1890]) and replated every 3-4 days at a split ratio of 1:10 following dissociation with Accutase (GE Healthcare, cat. L11-007) or 0.25% Trypsin (Life Technologies). Cells were cultured in serum-free N2B27-based medium (DMEM/F12 and Neurobasal in 1:1 ratio, 0.1 mM 2-mercaptoethanol, 2 mM L-glutamine, 1:200 N2 and 1:100 B27 [all reagents from Life Technologies]) or serum-containing KSR medium (GMEM [Sigma, cat. G5154] supplemented with 10% KSR [Life Technologies], 2% FBS [Sigma, cat. F7524], 100 mM 2-mercaptoethanol [Sigma, cat. M7522], 1×MEM non-essential amino acids [Invitrogen, cat. 1140-036], 2 mM L-glutamine, 1 mM sodium pyruvate [both from Invitrogen]), supplemented with two small-molecule inhibitors (2i) PD (1 μM, PD0325901), CH (3 μM, CHIR99021) from Axon (cat. 1386 and 1408) and LIF (100 units/ml purchased from Qkine—Cambridge UK).

Generation of HTT-expressing ES cell lines. Q15 and Q128 cells were generated by DNA transfection of vectors containing N-terminal of human huntingtin gene, with 128 or 15 CAG repeats respectively (courtesy of Professor Elena Cattaneo). Overnight linearization of plasmid DNA was performed with the restriction enzyme PvuI. For DNA transfection, we used Lipofectamine 2000 (Life Technologies, cat. 11668-019) and performed reverse transfection. For one well of a 6-well plate, we used 6 μl of transfection reagent, 2 μg of plasmid DNA, and 300,000 cells in 2 ml of medium. The medium was changed after overnight incubation. Antibiotic selection (Puromycin 1 μg/ml) started 24 h after transfection.

Generation of ES cells stably expressing genes of interest. Stable transgenic ESCs expressing candidates were generated by transfecting cells with PB transposon plasmids (1 μg of CAG-Mtf1, CAG-Kdm2b, CAG-Kdm5b and CAG-Fbxo34) with PB transposase expression vector pBase (1 μg). We used Lipofectamine 2000 and performed reverse transfection as described for HD lines generation. Antibiotic selection (Hygromycin B, 150 μg/ml; Invitrogen 10687010) started 24 h after transfection.

Proliferation assay. Cell proliferation was assessed by plating 15,000 ES cells in 24-well plate (7,500 cells/cm²) in presence of Puromycin 6 μg/ml. Cells were counted every 24 h for 4 days. Response to stressors was assessed by plating 5,000 ES cells in 24-well plate (2,500 cells/cm²) in the presence of the inhibitors (and Puromycin 6 μg/ml) for 48 h and scored by quantification of the number of surviving cells by Crystal Violet (CV) staining (CV solution: 0.05% w/v Crystal Violet [Sigma], 1% of formaldehyde solution 37% [Sigma], 1% methanol, 10% PBS). For PB-mutagenesis followed by stressor treatments, cells were plated at density 2,500 cells/cm² in Puromycin 6 μg/ml and selected for 5 days in the presence of MG132 or Tamoxifen.

Propidium iodide (PI) staining. PI staining was performed on live single ES cells according to the manufacturer's instructions (Ebioscience, cat. 88-8007-72). After washing in PBS, cells were resuspended in 100 μL of 1× Binding Buffer (cat. 00-0055) and incubated with 2.5 μL of fluorochrome-conjugated Annexin V (cat. 17-8007) for 10 minutes at room temperature. Cells were then washed once in PBS and resuspended in 100 μL of 1× Binding Buffer. Finally, 5 μL of Propidium Iodide Staining Solution (cat. 00-6990) were added to the cell suspension and flow cytometry analysis was performed within 1 hour, storing samples at 2-8° C. in the dark.

ROS measurement assay. Reactive oxygen species (ROS) production was detected by staining single live ES cells with 20,70-dichlorodihydrofluorescein diacetate (H2DCFDA) (Life Technologies, cat. D399), performing the following steps: a) shortly before performing the experiments, the ROS indicator was reconstituted in order to make a concentrated stock solution (10 mM); b) cells were harvested, c) washed once in PBS and d) resuspend in PBS containing the probe to provide a final working concentration of 0.5 μM dye; e) cells were incubated at 37° C. for 10 minutes in the dark; f) after removal of the staining solution, samples were g) washed twice in PBS and h) analyzed by flow cytometry using a BD FACSCanto II cytometer.

Electroporation of the PB system in ES cells. PB-mediated mutagenesis by electroporation was performed for genome-wide screening. PB vectors integrate stably in the genome after random insertion in TTAA sites. The PB pGG134 vector used (shown in FIG. 2a) was optimized for gain-of-function screens: it consists of the murine stem cell virus (MSCV) enhancer/promoter followed by a splice donor (SD) site, which allows the over-activation of nearby genes. The PB 5′ITR has also weak directional promoter activity, i.e. this construct can activate genes in either orientation. The vector contains also a second cassette, including a constitutive promoter followed by DsRed and Hygromycin resistance gene.

We optimized the conditions in order to achieve a low number of integration events, by adjusting the ratio of PB vector vs transposase pBase. For the screening procedure, mutagenesis was performed using the optimized amount of 0.5 μg pGG134 and 20 μg pBase. For a single electroporation, 10\hat{ }7 cells and 20.5 μg DNA were mixed and placed into an electroporation cuvette (Biorad Gene Pulser Cuvette, ca. 165-2088). Cells were electroporated by placing the cuvette in the electroporation holder of the Biorad GenePulser (ca. 165-2076). Settings used: 250V, 500 μF, time constant should be between 5.6 and 7.5. Electroporated cells were gently recovered from the cuvettes and plated. Antibiotic selection started 24 h after electroporation.

Genomic DNA extraction and Splinkerette-PCR. Cells were harvested and incubate o/n at 56° C. with lysis buffer (10 mM Tris, pH 7.5; 10 mM EDTA; 10 mM NaCl; 0.5% w/v Sarcosyl, supplemented with proteinase K [Sigma cat #P2308] to a final concentration of 1 mg/ml). In order to obtain DNA precipitates, the next day 2 ml of a mixture of NaCl and ethanol (30 μl of 5M NaCl mixed with 20 ml of cold absolute ethanol) was added. Cellular extracts were centrifuged for 45 min at 4° C. to remove soluble fraction. Precipitated gDNA was rinsed three times by dripping 2 ml of 70% ethanol and finally resuspended in 70° C.-milliQ water.

Splinkerette (Sp)-PCR procedure for PB-integration mapping was adapted from Potter and Luo, 2010 and consisted of the following steps: a) 2 μg of genomic DNA were digested with 10U BstYI (10,000 U/ml) in a volume of 30 μl. Reaction was incubated at 60° C. o/n, the following day the enzyme was inactivated at 80° C. for 20 min. Sp-adapters were generated by annealing of 150 pmol of AdapterA and B primers in a final volume of 100 μl (10×NEB Buffer 2). Oligos were denatured at 65° C. for 5 min, then cooled; b) Ligation was performed in a total volume of 6 μl including a 2× Ligation mix (Takara), 2.5 μl of digested gDNA and 0.5 μl Sp-adapters annealed. Ligation reaction was incubated at 16° C. o/n, the next day 65° C. for 10 min for enzyme inactivation. A purification step was included before step C, using QIaquick PCR Purification Kit, following manufacturer's instructions. For PCR amplifications we used Phusion HF DNA Pol (NEB) in 5× Phusion GC Buffer recommended in case GC-rich templates or those with secondary structures. PCR mix included 5×GC Buffer, 10 mM dNTPs, DMSO and Phusion Pol; c) First round PCR was amplified with 15 μl of ligated DNA (or 50% of ligation product for each reaction for PB5′ and PB3′ transposon/host junctions), 0.5 μM for each primer (Adapter-PCR1 and PB5′ or PB3′-ITR PCR1), 6.5 μl PCR mix, final volume of 25 μl. Sp-PCR1 program: 95° C. for 2 min; two cycles of 95° C. for 20 sec, 65° C. for 30 sec, 68° C. for 2 min; then 30 cycles of 95° C. for 30 sec, 60° C. for 30 sec, 68° C. for 2 min; then 68° C. for 10 min; d) For second round PCR, we used 5 μl of 1:500 dilution of PCR1 product, 0.5 μM for each primer (Adapter-PCR2 and PB5′ or PB3′-ITR PCR2), 6.5 μl PCR mix, final volume of 25 μl. Sp-PCR2 program: 95° C. for 2 min; two cycles of 95° C. for 20 sec, 65° C. for 30 sec, 68° C. for 2 min; then 5 cycles of 95° C. for 30 sec, 60° C. for 30 sec, 68° C. for 2 min; then 25 cycles of 95° C. for 30 sec, 58° C. for 30 sec, 68° C. for 2 min; then 68° C. for 10 min; e) PCR2 products were treated with Antarctic Phosphatase and Exonuclease I (both from NEB) and sequenced using PB5′ or PB3′-ITR PCR2 primers. Primers and adaptor sequences are listed in Table 1 of the paper from Potter and Luo, 2010.

Next Generation Sequencing analysis of genomic integration sites. Genomic DNA from entire populations of mutants was extracted using a Gentra Puregene Cell Kit. Library preparation and sequencing was performed as described in Lackner et al., 2020 (bioRxiv). A bespoke bioinformatics pipeline allowed to map each single read to a genomic locus and to associate each site of integration to a gene within 20 kb of distance. Data were then organized into the network of HD interacting genes by means of Cytoscape software.

RNA isolation, reverse transcription and quantitative PCR. For cellular lysate, RNA was isolated using Total RNA Purification Kit (Norgen Biotek 37500), and complementary DNA (cDNA) was made from 1 μg using M-MLV reverse transcriptase (Invitrogen 28025-013) and oligodT18 (500 μg/ml) primers. For zebrafish larvae, total RNA was isolated taking advantage of the phenol-chloroform extraction. Total RNA was isolated from pools of 10 animals by using TRIzol Reagent (Life Technologies, cat. no. 15596026), following manufacturer's instructions for standard trizol-chloroform-ethanol extraction procedure. RNase-free glycogen was used as suggested by the protocol, to increase the yield of the RNA precipitation step. 2 μg of total RNA were reverse transcribed into cDNA by using Superscript III Reverse Transcriptase (Invitrogen, cat. no. 18080044) and a mixture of: oligodT18 primers (500 μg/ml); dNTP mix (10 mM); DTT (0.1M); 5× First-Strand Buffer; RNaseOUT (40 units/μl).

For real-time PCR, SYBR Green Master mix (Bioline BIO-94020) was used. The primers used are reported in Table 5 above. Technical replicates were carried out for all quantitative PCR. For ESCs, Gapdh was used as endogenous control to normalise expression. The Ct mean of zebrafish gapdh, eef1a, tuba1b and b2m was used as an endogenous housekeeping control for normalization, due to the variability shown looking at the expression levels of those genes. qPCR data were acquired with QuantStudio™ 6&7 Flex Software 1.0.

Western Blotting. Cells were washed in cold PBS and harvested in lysis buffer (50 mM Hepes pH 7.8, 200 mM NaCl, 5 mM EDTA, 1% NP40, 5% glycerol), freshly supplemented with 1 mM DTT, protease inhibitor (Roche 39802300) and phosphatase inhibitor (Sigma-Aldrich P5726). Samples were exposed to ultrasound in a sonicator (Diagenode Bioruptor) and centrifuged at 13000 rpm for 10 minutes to prepare supernatant. Protein concentration was determined by Bradford quantification. Total protein (10 μg) was fractionated on 4-12% Nupage MOPS acrylamide gel (Life Technologies; BG04125BOX/BG00105BOX) and electrophoretically transferred on a PVDF membrane (Millipore; IPFL00010) in a Transfer solution (50 mM Tris, 40 mM glycine, 20% methanol, 0.04% SDS).

Membranes were then saturated with 5% Non-Fat Dry Milk powder (BioRad; 170-6405-MSDS) in TBSt (8 g NaCl, 2.4 g Tris, 0.1% Tween20/liter, pH 7.5) for 1 hour at RT and incubated overnight at 4° C. with HTT or GAPDH primary antibody (Millipore cat. MAB2166 and Millipore cat. MAB374). Mice were sacrificed by cervical dislocation and tissues were homogenized in lysis buffer containing 20 mM Tris, pH 7.4, 1% Nonidet P-40, 1 mM EDTA, 20 mM NaF, 2 mM Na3VO4 and 1:1000 protease inhibitor mixture (Sigma-Aldrich) and sonicated. 40 μg of total protein lysate were immunoblotted with the following antibodies: anti-GFP and anti-Actin (Cell Signaling Technology cat. 3700). Membranes were then incubated with secondary antibodies conjugated with a peroxidase, diluted in 1% milk in TBSt. Pico SuperSignal West chemiluminescent reagent (Thermo Scientific; 34078) was used to incubate membranes and images were digitally acquired by ImageQuant LAS 4000 (GE Healthcare).

RNA sequencing: Library Preparation. Total RNA was quantified using the Qubit 2.0 fluorimetric Assay (Thermo Fisher Scientific). Libraries were prepared from (125*-250**-250***) ng of total RNA using a 3′DGE mRNA-seq (research*/clinical**/diagnostic***) grade sequencing service (Next Generation Diagnostics srl) which included library preparation, quality assessment and sequencing on a NovaSeq 6000 sequencing system using a single-end, 100 cycle strategy (Illumina Inc.).

Bioinformatics workflow: The raw data were analyzed by Next Generation Diagnostics srl proprietary 3′DGE mRNA-seq pipeline (v2.0) which involves a cleaning step by quality filtering and trimming, alignment to the reference genome and counting by gene. We have filtered out all genes having <1 cpm in less than n_min samples and Perc MM reads >20% simultaneously. Differential expression analysis was carried out in R environment (v. 3.5.3) with Bioconductor (v.3.7) exploiting the DESeq2 R package. (v. 1.24.0) and edgeR. DESeq2 performs the estimation of size therapeutic proteins, the estimation of dispersion for each gene and fits a negative binomial generalized linear model with Wald statistics. Genes with p-value >=0.05 between Q128_Mtf1 and Q15_EV and log 2FC>|0.5| between Q128_Mtf1 and Q128_EV were considered significant and defined as differentially expressed (DEGs).

Gene set enrichment analysis of DEGs was performed using Database for Annotation, Visualisation and Integrated Discovery (DAVID) database (https://david.ncifcrf.gov) exploiting UniProt Keywords database.

Volcano plots were produced with Log 2 fold-change and −Log 10 p-value exploiting ggscatter function from ggpubr R package (v. 0.2.5). Heatmaps were made using CPM values with pheatmap function from pheatmap R package (v.1.0.12).

Generation of Zebrafish HD model. HD zebrafish were generated by microinjection in one-cell stage embryos of mRNA encoding the first exon of human HTT including 74Q or 16Q, fused in-frame to eGFP coding sequence. Q74eGFP and Q16eGFP were cloned into pCS2+ plasmid in order to allow for the in vitro transcription. pCS2_Mtf1 and pCS2_mCherry plasmids were also generated to obtain Mtf1 and mCherry mRNAs used for injections in HD zebrafish embryos.

For RNA in vitro transcription, 2.5 μg of pCS2_Q74eGFP, pCS2_Q16eGFP, pCS2_Mtf1, and pCS2_mCherry were linearized by overnight digestion at 37° C. with HF-Not I (New England Biolabs, cat. no. R3189S). The digestion volume was then concentrated by the DNA Clean & Concentrator kit (Zymo Research, cat no. D4003) and used for the capped transcription reaction (mMESSAGE mMACHINE™ SP6 Transcription Kit, Thermo Fisher Scientific, cat. no. AM1340) by SP6 RNA polymerase. After removing the DNA template by DNase treatment (Thermo Fisher Scientific, cat. no. AM2238) for 15 minutes at 37° C., RNA was purified by Phenol-Chloroform extraction (as discussed in ‘RNA isolation, reverse transcription and quantitative PCR’ paragraph). RNA was quantified by Nanodrop ND-1000 and then diluted according to the need in a mix of 10% Danieau buffer (8 mM NaCl, 0.7 Mm KCl, 0.4 mM MgSO4, 0.6 mM Ca(NO3)2, 2.5 mM HEPES, Ph 7.6), 10% Phenol Red (Merck, cat. no. 1072410025) and RNase-free water.

In order to select the injection dose that caused the highest rate of malformations with the lowest level of death, we injected increasing doses of Q74eGFP mRNA ranging from 150 to 1000 pg/embryo and phenotypically scored 24 hpf embryos. Once established the dose of 250 pg/embryo, under a light microscope, embryos were injected with transcribed mRNAs. Microinjected embryos were then transferred to fish water and incubated at 28° C. Unfertilized eggs were recognised and discarded 4 hours post-microinjection. 24 hpf tadpoles were dechorionated using dedicated needles under a light microscope.

Whole-mount stainings. Injected embryos were anesthetised with tricaine and immobilized in 1.5% Methylcellulose or 2% low melting agarose and analysed using a Leica M165FC fluorescence microscope. Confocal zebrafish images were acquired with a Nikon C2 H600L confocal microscope.

For Acridine Orange hemi (zinc chloride) in vivo staining (Merck, cat. No. A6014), 24 hpf embryos were dechorionated, transferred into a 6-well plate and incubated in about 2 ml of Acridine Orange (20 μg/ml) per well in fish water for 15 minutes at 28° C. The Acridine solution was then removed and embryos were washed three times with 1 ml of fish water. Before being observed on a glass slide by a fluorescence microscope, tadpoles were anesthetized by Tricaine.

For the TUNEL assay, ApopTag Fluorescein In Situ Apoptosis Detection Kit (Merck, cat. no. S7110) and collagenase (Merck, cat. no. C9891) were used. 7 embryos—30 h post microinjection-per condition were placed in an Eppendorf, anesthetised with Tricaine and fixed in 4% paraformaldehyde (PFA) at 4° C. overnight. Then, PFA was removed and samples were washed 3 times with Phosphate-buffered saline; 10 minutes each, while shaking. Embryos were dehydrated through a series methanol solutions ranging from 10% to 100% and frozen at −20° C. overnight. Then, embryos were rehydrated with a series of 70-50-30% methanol solutions and washed by PBS with Tween-20 (PBST), 10 minutes per wash, while shaking. After that, collagenase was applied for 8 minutes while shaking and the excess was washed away by 3 PBST washing steps of 5 minutes. Samples were incubated for 1 hour in the equilibration buffer while shaking, then for 2 hours at 37° C. in working strength TdT. The reaction was stopped by washing twice the samples in the working strength Stop/Wash buffer. Next, there was a blocking step of 1 hour with PBST while shaking, and then embryos were incubated overnight in working strength anti-digoxigenin conjugate at 4° C. in the dark. The morning after, the antibody solution was removed, samples were washed with PBS (4 times, 10 minutes each) and analysed by a confocal microscope.

Image analysis. For quantification analyses, all images were acquired with the same exposure parameters and processed using FIJI software. Statistical analyses were carried out with Past4 software.

For the TUNEL assay, Zebrafish larvae anterior structures were scanned in 70 stacks of 3.475 μm each, spanning their entire depth. We quantified the fractions of fluorescent positive area over the total area (excluding the yolk region).

### AAV-PHP.eB Vector Injection, Mouse Phenotyping and Tissue Collection

AAV-PHP.eB viral particles were produced and titered in Broccoli's lab as described in Morabito et al., 2017. This viral vector has been modified to express under the control of the Ef-1α promoter the candidate gene Mtf1 or either eGFP as a control. Vascular injection was performed in a restrainer that positioned the tail in a heated groove. The tail was swabbed with alcohol and then injected intravenously. WT and R6/2 mice were randomized in groups and injected in the tail vein at 4.2 weeks of age. Following injection, all mice were weighed twice a week. Phenotyping was carried out, blind to genotype and treatment, twice a week. The balance and the motor coordination were assessed by Rotarod test and Horizontal Ladder Task. Total DNA was isolated from animal tissues (cortex and striatum) using the Qiagen DNeasy Blood and Tissue Kits (QIAGEN).

Animal husbandry. All Zebrafish experiments were carried out at the Fish Facility in the Department of Biology of the University of Padova. Zebrafish larvae were kept at most three days in Petri dishes with fish water (60 mg of Instant Ocean, cat. no. SS15-10, per litre of distilled water) at neutral pH at 28° C., according to standard procedures (http://ZFIN.org). Mice were maintained at IRCCS Neuromed Institute institutional mouse facility (Pozzilli, Italy) in micro-isolators under sterile conditions and supplied with autoclaved food and water. Breeding pairs of the R6/2 line of transgenic female mice [strain name: B6CBA-tgN (HDexon1) 62Gpb/1J] with ˜160±10 (CAG) repeat expansions were purchased from the Jackson Laboratories. Male R6/2 mice were crossed with female B6CBA WT mice for colony maintenance. All procedures were performed according to protocols approved by the internal institutional animal care and use committee (IACUC) and reported to the Italian Ministry of Health according to the European Commission Council Directive 2010/63/EU and to Italian legislation on animal experimentation (Decreto Legislativo D.Lgs 26/2014).

1. A method of reducing toxicity of a mutated protein in a subject in need thereof, said mutated protein causing a polyQ disease, said method comprising
administering to said subject a therapeutic effective amount of a therapeutic protein, or an isoform or a homolog thereof, or of an mRNA coding for said therapeutic protein, isoform or homolog thereof, or a pharmaceutical composition comprising said therapeutic protein or said mRNA, said homolog having a sequence homology, defined as at least 55% coverage and at least 49% identity with said therapeutic protein.

2. The method according to claim 1 wherein said mutated protein is expressed by one or more of ATN1, HTT, AR, ATXN1, ATXN2, ATXN3, CACNA1A, ATXN7, PPP2R2B, TBP genes, said mutated protein comprising a pathological number of polyQ residues.

3. The method according to claim 1, wherein said therapeutic protein is selected from metal regulatory transcription factor 1, lysine-specific demethylase 5B, lysine-specific demethylase 2B, F-box only protein 34, essential MCU regulator, mitochondrial, Ephrin type-A receptor 4, Transducin-like enhancer protein 4, Arfaptin-1, and AT-rich interactive domain-containing protein 5B.

4. The method according to claim 1, wherein said isoform is selected from Table 3 below

| Therapeutic protein | |
|---|---|
| from table 2 | Isoforms |
| MTF1 | NM_005955.3; XM_011541494.1; XM_011541491.2; XM_011541493.3 |
| KDM5B | NM_001314042.1; NM_001347591.1; NM_006618.5 |
| KDM2B | NM_001005366.2; NM_032590.5; XM_011538867.3; XM_011538868.3; |
| | XM_005253955.4; XM_005253956.4; XM_005253961.5; XM_011538875.3; |
| | XM_011538869.2 |
| FBX034 | NM_017943.4; NM_152231.2; XM_006720185.3; XM_017021391.1; |
| | XM_017021392.1; XM_017021393.2; XM_005267791.4 |
| SMDT1 | NM_033318.5; NR_146715.2; XM_011530509.3; NR_146717.1 |
| EPHA4 | NM_001304536.2; NM_001304537.2; NM_001363748.2; NM_004438.5 |
| TLE4 | NM_001282748.2; NM_001282749.2; NM_001282753.2; NM_001282760.2; |
| | NM_001351541.2; NM_001351542.2; NM_001351543.2; NM_001351546.2; |
| | NM_001351547.2; NM_001351550.2; NM_001351552.2; NM_001351556.2; |
| | NM_001351558.2; NM_001351560.2; NM_001351562.2; NM_001351563.2; |
| | NM_001351564.2; NM_007005.6; NR_104239.2; XM_011518952.1; |
| | XM_006717264.1; XM_011518958.1; XM_006717268.1; XM_011518967.1; |
| | XM_011518969.1; XM_011518956.1; XM_011518957.1; XM_011518959.1; |
| | XM_011518965.1; XM_017015081.1; XM_011518960.1; XM_011518966.1; |
| | XM_017015083.1; XM_011518963.1; XM_011518964.1; XM_011518955.1; |
| | XM_011518961.1; XM_011518962.1; XM_011518968.1; XM_011518954.1; |
| | XM_017015068.1; XM_017015075.1; XM_011518970.3; XM_017015070.1; |
| | XM_017015067.1; XM_011518953.2; XM_017015069.2; XM_011518972.3; |
| | XM_024447659.1 |
| ARFIP1 | NM_001025593.3; NM_001025595.3; NM_001287431.2; NM_001287432.2; |
| | NM_001287433.2; NM_014447.4 |
| ARID5B | NM_001244638.2; NM_032199.3 |

5. The method according to claim 1, wherein said homolog is selected from Table 4 below

| Homolog | REFERENCE NCBI |
|---|---|
| KDM5A | 5927 |
| KDM5C isoform 3 | NP_001269551.1 |
| FBXL11 | 22992 |
| EPHA5 isoform d and b | NP_001268695.1 |
| | NP_872272.2 |
| EPHA5 isoform a and c | NP_004430.4 |
| | NP_001268694.1 |
| EPHA7 isoform 2 precursor | NP_001275558.1 |
| EPHA7 isoform 5 precursor | NP_001363395.1 |
| EPHA7 isoform 1 precursor | NP_004431.1 |
| EPHA7 isoform 4 precursor | NP_001363394.1 |
| EPHA5 isoform f | NP_001305690.1 |
| EPHA5 isoform e | NP_001268696.1 |
| EPHA3 isoform a precursor | NP_005224.2 |
| EPHA3 isoform X1 | XP_005264772.1 |
| TLE3 isoform e | NP_001269908.1 |
| TLE3 isoform X5 | XP_005254680.1 |
| TLE3 isoform d | NP_001269909.1 |
| TLE3 isoform b | NP_001098662.1 |
| TLE3 isoform X7 | XP_011520282.1 |
| TLE3 isoform X4 | XP_011520280.1 |
| TLE3 isoform X3 | XP_005254679.1 |
| TLE3 isoform a | NP_005069.2 |
| TLE3 isoform X2 | XP_011520279.1 |
| TLE3 isoform X1 | XP_011520278.1 |
| TLE3 isoform f | NP_001269910.1 |
| TLE3 isoform X15 | XP_005254690.1 |
| TLE3 isoform X10 | XP_005254685.1 |
| TLE3 isoform c | NP_065959.1 |
| TLE3 isoform X8 | XP_005254682.1 |
| TLE1 isoform X12 | XP_006717323.1 |
| ARFIP2 isoform 9 | NP_001363487.1 |
| ARFIP2 isoform 5 | NP_001357335.1 |
| ARFIP2 isoform 10 | NP_001363489.1 |
| ARFIP2 isoform 1 | NP_001229783.1 |
| ARFIP2 isoform 8 | NP_001357341.1 |
| ARFIP2 isoform 7 | NP_001357338.1 |

6. The method according to claim 1, wherein said polyQ disease is Dentatorubropallidoluysian atrophy, Huntington's disease, Spinal and bulbar muscular atrophy, Spinocerebellar ataxia Type 1, Spinocerebellar ataxia Type 2, Spinocerebellar ataxia Type 3, Spinocerebellar ataxia Type 6, Spinocerebellar ataxia Type 7, Spinocerebellar ataxia Type 12 or Spinocerebellar ataxia Type 17.

7.-8. (canceled)

9. A method of treating a polyQ disease in a subject in need thereof, said method comprising
administering to said subject a therapeutically effective amount of an expression vector or delivery system or vector suitable for gene therapy, or adeno-associated viral vector AAV or Lentiviral vector, or nanoparticle, or LNP or nucleotide sequence coding for the therapeutic protein as defined in claim 1.

10. The method according to claim 9 wherein said polyQ disease is Dentatorubropallidoluysian atrophy, Huntington's disease, Spinal and bulbar muscular atrophy, Spinocerebellar ataxia Type 1, Spinocerebellar ataxia Type 2, Spinocerebellar ataxia Type 3, Spinocerebellar ataxia Type 6, Spinocerebellar ataxia Type 7, Spinocerebellar ataxia Type 12 or Spinocerebellar ataxia Type 17.

11. A method of reducing toxicity of a mutated protein in a subject in need thereof, said mutated protein causing a polyQ disease, said method comprising
administering to said subject a therapeutic effective amount of a viral particle comprising a viral capsid and a nucleotide sequence coding for the therapeutic protein or isoform or homolog thereof as defined in claim 1.

12. (canceled)

13. The method according to claim 11 wherein said said polyQ disease is Dentatorubropallidoluysian atrophy, Huntington's disease, Spinal and bulbar muscular atrophy, Spinocerebellar ataxia Type 1, Spinocerebellar ataxia Type 2, Spinocerebellar ataxia Type 3, Spinocerebellar ataxia Type 6, Spinocerebellar ataxia Type 7, Spinocerebellar ataxia Type 12 or Spinocerebellar ataxia Type 17.

14. (canceled)

15. The method according to claim 14, wherein said mRNA is complexed with one or more carrier molecules.

16. The method according to claim 15, wherein said mRNA is complexed in a cationic nanoemulsion, in a nanoparticle, in a liposome, in a cationic polymer liposome, in a polysaccharide particle, in a cationic lipid nanoparticle, in a cationic lipid cholesterol nanoparticle, in a cationic lipid cholesterol PEG nanoparticle.

17. (canceled)

18. The method according to claim 14, wherein said polyQ disease is Dentatorubropallidoluysian atrophy, Huntington's disease, Spinal and bulbar muscular atrophy, Spinocerebellar ataxia Type 1, Spinocerebellar ataxia Type 2, Spinocerebellar ataxia Type 3, Spinocerebellar ataxia Type 6, Spinocerebellar ataxia Type 7, Spinocerebellar ataxia Type 12 or Spinocerebellar ataxia Type 17.

19.-21. (canceled)

22. The method according to claim 9, wherein said vector is a viral vector AAV having capsid serotype for CNS gene therapy.
